(19) United States
(12) Patent Application Publication
(10) Pub. No.: US 2024/0420843 A1
(43) Pub. Date: Dec. 19, 2024

(54) METAEPIGENOMICS-BASED DISEASE DIAGNOSTICS

(71) Applicant: Micronoma, Inc., San Diego, CA (US)

(72) Inventors: Eddie Adams, San Diego, CA (US); Stephen Wandro, San Diego, CA (US); Serena Fraraccio, San Diego, CA (US); Akanksha Singh-Taylor, San Diego, CA (US)

(21) Appl. No.: 18/698,916

(22) PCT Filed: Oct. 7, 2022
(86) PCT No.: PCT/US2022/046126

§ 371 Date: Apr. 5, 2024

Related U.S. Application Data
(60) Provisional application No. 63/253,655, filed on Oct. 8, 2021.

(51) Int. Cl. G16H 50/20; C12Q 1/6806; C12Q 1/6886; G16B 20/00; G16B 40/20

(52) U.S. Cl. CPC G16H 50/20; C12Q 1/6806; C12Q 1/6886; G16B 20/00; G16B 40/20; C12Q 2600/154

(57) ABSTRACT

Provided are methods for creating a diagnostic model for determining a disease of a subject, methods to identify disease-associated metaepigenomic biomarkers and methods to employ these biomarkers to accurately diagnose certain diseases from a tissue or liquid biopsy sample, based on epigenetic data from the subject's genome and microbial genomes contained within that subject

### CROSS-REFERENCE

This application claims the benefit of U.S. Provisional Application No. 63/253,655 filed Oct. 8, 2021, which application is incorporated herein by reference.

### SUMMARY

The disclosure of the present invention provides methods to identify disease-associated metaepigenomic biomarkers and methods to employ these biomarkers to accurately diagnose certain diseases from a tissue or liquid biopsy sample. Specifically, the present invention provides methods for enriching and integrating inter-kingdom epigenetic data derived from the mammalian, bacterial, fungal, archaeal, and viral kingdoms of life from a tissue or liquid biopsy sample and methods for using this combined dataset to diagnose and classify disease in a mammalian subject.

The methods of the present invention disclosed herein provide a means of discovering disease-diagnostic biomarkers from inter-kingdom nucleic acid analyses, wherein the biomarkers specifically derive from epigenetic features contained within a mixed (i.e., multi-kingdom) population of nucleic acids. These epigenetic features may be, for instance, a common feature shared by two or more taxonomic kingdoms or may be taxonomically divergent, non-overlapping epigenetic features that are independently analyzed and thereafter combined to provide an inter-kingdom diagnostic signature.

Human DNA methylation-based biomarkers have long been the subject of academic and clinical research (see, for example, DNA Methylation and Complex Human Disease, Michael Neidhart, 2016, ISBN: 978-0-12-420194-1) and have been incorporated into several commercial diagnostic assays that utilize the disease-characteristic presence or absence of 5-methylcytosine (5mC)-modified DNA. For example, the only blood-based liquid biopsy assay that has obtained FDA approval for cancer diagnosis is Epigenomics's Epi proColon colorectal cancer screening assay. This is a PCR assay for the qualitative detection of methylated Septin9 ctDNA isolated from 3.5 milliliters of patient plasma (methylation of certain CpG motifs in the promoter region of the SEPT9_v2 transcript has been associated with colorectal cancer but not healthy tissue). Specifically, the Epigenomics assay utilizes bisulfite-treatment of isolated cfDNA and methylation-specific primers to detect the presence of methylated Septin9. More recently, Grail Inc, used differential DNA methylation of genomic CpG sites to discriminate among different cancers and cancer versus non-cancer samples. GRAIL has set an ambitious goal to accurately screen for more than 50 unique cancer types from a single sample through targeted bisulfite sequencing analysis of cell-free circulating tumor DNA (ctDNA) methylation patterns. DNA methylation-based biomarkers have been explored in many disease areas but may prove particularly useful in liquid biopsy-based cancer diagnostics as a means of determining which ctDNA fragments are truly tumor-derived. While most driver mutations in oncogenes (e.g., TP53, KRAS) are common among cancers regardless of their tissue of origin, CpG methylation profiles are highly specific to tissues and tumors-derived therefrom, potentially enabling a more exact diagnosis of cancer. In addition, there are 28 million CpG sites throughout the human genome whose methylation states (methylated versus unmethylated) may comprise a cancer-specific signature whereas canonical ctDNA mutations are limited in copy number/genome and therefore impose a sensitivity limitation for detection. In these and other analyses utilizing mammalian DNA modifications it is important to emphasize that these epigenetic analyses are conducted with the deliberate exclusion of nucleic acid data from non-mammalian sources, which may be concurrently evident in a disease-specific presence or abundance.

Likewise, while it is appreciated that microbial genomes harbor epigenetic information in the form of heritable yet enzymatically reversible chemical modifications of the genome's underlying polynucleotide sequences, differences between mammalian and microbial DNA methylation have hitherto been used as a means of separating prokaryotic DNA from mammalian DNA to improve the diagnostic sensitivity of assays focused on select prokaryotic targets. For instance, Schmidt et al. (U.S. Pat. No. 8,288,115 B2) teaches the use of certain proteins (Toll-like receptor 9 (TLR9), and CpG-binding protein (CGBP)) to enrich non-methylated prokaryotic DNA from a sample containing both mammalian and non-mammalian DNA. As unmethylated CpG sites are 20 times more abundant in prokaryotic DNA than mammalian DNA, physical enrichment of unmethylated CpG-containing DNA serves to limit the amount of mammalian DNA present in downstream molecular assays, specifically, per Schmidt et al., PCR-based analysis.

In a similar vein, Forsyth (U.S. Pat. No. 8,927,218 B2) teaches the use of catalytically inactive restriction enzymes capable of binding, but not hydrolyzing, specific microbial DNA methylation motifs, and methylation-specific antibodies to concentrate prokaryotic sequences from complex mixtures of nucleic acids. Here again the intent is to physically separate prokaryotic sequences from non-prokaryotic sequences such that downstream analyses focused on detection of select prokaryotes gain improved limits of detection.

Zhou et al. (WO 2020/198664; PCT US2020/025425) teach a method of preparing sequencing libraries from cell-free DNA to facilitate genomic and epigenomic profiling of microbiome' but, here again, the aim is to separate mammalian nucleic acid molecules from non-mammalian such that most downstream sequencing reads are of microbial origin. Furthermore, while the method of Zhou et al, provides a means of preparing sequencing libraries that may be amenable to microbial epigenomic analyses, the manner of epigenomic analysis or the epigenetic features to be analyzed are not taught.

In contrast to the foregoing art wherein epigenetic features of exclusively mammalian or non-mammalian origin—but not of both—are subject to analysis, the methods of the present invention harness and combine the epigenetic data derived from taxonomically diverse life forms manifest within a nucleic acid sample. As microbes are increasingly implicated in mammalian disease processes and disease-specific mammalian epigenetic features have proven a robust source of diagnostic biomarkers, we reasoned that combining the epigenetic content from both mammalian and microbial taxonomic sources within a nucleic acid sample would enable the creation of highly sensitive and specific metaepigenomic diagnostic signatures. In this manner we diverge sharply from all existing art and produce a novel method of identifying disease-diagnostic biomarkers.

Aspects disclosed herein provide a method of creating a diagnostic model for diagnosing disease in a subject based on the combination of mammalian and non-mammalian epigenetic information contained in a nucleic acid sample, comprising: (a) enriching one or more mammalian and non-mammalian nucleic acid molecules by affinity targeting of an epigenetic feature shared by both the one or more mammalian and non-mammalian nucleic acid molecules; (b) sequencing the enriched nucleic acid compositions to generate sequencing reads; (c) filtering the sequencing reads with a build of a genome database to isolate non-mammalian sequencing reads and produce a mammalian alignment file; (d) analyzing the mammalian alignment file to generate mammalian feature abundance tables; (e) analyzing the non-mammalian sequencing reads to generate non-mammalian feature abundance tables; (f) combining the mammalian and non-mammalian feature abundance tables to generate combined meta-epigenomic machine learning feature sets; (g) training and testing predictive models on the meta-epigenomic feature sets to produce a trained predictive model; and (h) using an output of the trained predictive model to provide a diagnosis of a presence or absence of the disease in the subject. In some embodiments, the nucleic acid sample may be derived from a tissue, liquid biopsy sample or any combination thereof. In some embodiments, the subject may be a human or a non-human mammal. In some embodiments, the nucleic acids may comprise a total population of DNA, RNA, cell-free DNA, cell-free RNA, exosomal DNA, exosomal RNA or any combination thereof.

In some embodiments, affinity targeting may comprise concentrating a shared nucleic acid epigenetic feature. In some embodiments, the shared nucleic acid epigenetic feature may comprise methylated CpG dinucleotides pairs. In some embodiments, the shared nucleic acid epigenetic feature may comprise unmethylated CpG dinucleotide pairs. In some embodiments, the shared nucleic acid epigenetic feature may comprise the modified nucleobases 5-methylcytosine, 5-hydroxymethylcytosine, N4-acetylcytosine, and N6-methyladenine.

In some embodiments, the affinity targeting may comprise specific affinity reagents. In some embodiments, the specific affinity reagents may comprise streptavidin, NeutrAvidin, polyclonal, monoclonal, recombinant antibodies, aptamers, or recombinant epigenetic proteins. In some embodiments, the recombinant epigenetic proteins may comprise epigenetic readers, writers, erasers, or any combination thereof. In some embodiments, the epigenetic readers may comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom. In some embodiments, the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. In some embodiments, the epigenetic writers and erasers may be catalytically inactive. In some embodiments, the epigenetic readers, writers, and erasers may comprise an epitope tag. In some embodiments, the epitope tag may comprise an N- or C-terminal 6x-histidine tag, green fluorescent protein (MA), myc, hemagglutinin (HA), Fc fusion, molecular recognition motif or any combination thereof. In some embodiments, the molecular recognition motif may comprise a birA or sortase motif. In some embodiments, the nucleic acid compositions may be concentrated by a solid support, wherein the solid support may comprise covalently bonded complementary antibodies to the epitope tag. In some embodiments, the specific affinity reagents may comprise a region to recognize and bind to the epigenetic feature. In some embodiments, the affinity targeting may comprise incubating the nucleic acid sample with a solid support comprising a plurality of immobilized affinity agents. In some embodiments, the plurality of immobilized affinity agents may comprise a region that will bind to the epigenetic feature. In some embodiments, the solid support may comprise a magnetic bead, an agarose bead, non-magnetic latex, functionalized Sepharose, pH-sensitive polymers or any combinations thereof. In some embodiments, the genome database may be a human genome database.

In some embodiments, the mammalian feature abundance tables may comprise mammalian genomic coordinates or annotated genomic loci and the number of sequencing reads associated therewith. In some embodiments, the mammalian feature abundance tables may comprise mammalian functional gene and biochemical pathway abundance tables. In some embodiments, the non-mammalian feature abundance tables may comprise microbial taxonomic assignments and the number of sequencing reads associated therewith. In some embodiments, the non-mammalian features abundance tables may comprise non-mammalian functional gene and biochemical pathway abundance tables. In some embodiments, the output of the trained predictive model may comprise an analysis of a combination of the mammalian and non-mammalian feature sets. In some embodiments, the trained predictive model may be trained with a set of mammalian and non-mammalian epigenomic abundances that are known to be present with a characteristic abundance or absent in a disease of interest. In some embodiments, the diagnostic model may utilize epigenomic abundance information from one or more of the following kingdoms of life: mammalian, bacterial, archaeal, fungal, and/or viral. In some embodiments, the diagnostic model may diagnose a category or tissue-specific location of disease. In some embodiments, the diagnostic model may be used to diagnose one or more types of cancer in a subject. In some embodiments, the diagnostic model may be used to diagnose one or more subtypes of cancer in a subject. In some embodiments, the diagnostic model may be used to predict the stage of cancer in a subject and/or predict cancer prognosis in the subject. In some embodiments, the diagnostic model may be used to predict cancer therapy response of the subject. In some embodiments, the diagnostic model may be utilized to select an optimal therapy for a particular subject. In some embodiments, the diagnostic model may be utilized to longitudinally model a course of one or more cancers' response to a therapy and to then adjust a treatment regimen.

In some embodiments, the diagnostic model may diagnose one or more of the following: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum cell tumors, thymoma, thyroid carcinoma, uterine carcinosarcoma, uterine corpus endometrial carcinoma, or uveal melanoma. In some embodiments, the diagnostic model may identify and remove certain non-human features as contaminants termed noise, while selectively retaining other non-human features termed signal. In some embodiments, the diagnostic model may be used to diagnose systemic lupus erythematosus, type 2 diabetes, chronic obstructive pulmonary disease (COPD), or sarcoidosis. In some embodiments, the liquid biopsy sample may include but is not limited to one or more of the following: plasma, serum, whole blood, urine, cerebral spinal fluid, saliva sweat, tears, or exhaled breath condensate.

Aspects disclosed herein provide a method of creating a diagnostic model for diagnosing disease in a subject based on the combination of mammalian and non-mammalian epigenetic information contained in a nucleic acid sample, comprising (a) enriching one or more mammalian nucleic acid molecules by affinity targeting of an epigenetic feature present in one or more mammalian nucleic acid molecules; (b) enriching one or more non-mammalian nucleic acid molecules by affinity targeting of an epigenetic feature present in one or more non-mammalian nucleic acid molecules; (c) sequencing the enriched mammalian nucleic acid compositions to generate sequencing reads; (d) sequencing the enriched non-mammalian nucleic acid compositions to generate sequencing reads; (e) aligning the mammalian sequencing reads to a build of a genome database to produce a mammalian alignment file; (f) filtering the non-mammalian sequencing reads with a build of a genome database to isolate non-mammalian sequencing reads; (g) analyzing the mammalian alignment file to generate mammalian feature abundance tables; (h) analyzing the non-mammalian sequencing reads to generate non-mammalian feature abundance tables; (i) combining the mammalian and non-mammalian feature abundance tables to generate combined meta-epigenomic machine learning feature sets; (j) training and testing predictive models on the meta-epigenomic feature sets to produce a trained predictive model; and (k) using an output of the trained predictive model to provide a diagnosis of a presence or absence of the disease in the subject. In some embodiments, the nucleic acid sample may be derived from a tissue, liquid biopsy sample or any combination thereof. In some embodiments, the subject may be a human or a non-human mammal. In some embodiments, the nucleic acids may comprise a total population of DNA, RNA, cell-free DNA, cell-free RNA, exosomal DNA, exosomal RNA or any combination thereof.

In some embodiments, affinity targeting may comprise concentrating mammalian and non-mammalian nucleic acid epigenetic features. In some embodiments, the mammalian nucleic acid epigenetic features may comprise the modified nucleobases 5-methylcytosine, 5-hydroxymethylcytosine, 5-formylcytosine, 5-carboxycytosine, N4-acetylcytosine, and N6-methyladenine. In some embodiments, the non-mammalian nucleic acid epigenetic features may comprise the modified nucleobases 5-methylcytosine, 5-hydroxymethylcytosine, 4-methylcytosine, N4-acetylcytosine, N6-methyladenine. In some embodiments, the non-mammalian nucleic acid epigenetic feature may comprise phosphorothioate-linked nucleotides.

In some embodiments, affinity targeting may comprise specific affinity reagents. In some embodiments, the specific affinity reagents may comprise streptavidin, NeutrAvidin, polyclonal, monoclonal, recombinant antibodies, aptamers, or recombinant epigenetic proteins. In some embodiments, the recombinant epigenetic proteins may comprise epigenetic readers, writers, erasers, or any combination thereof. In some embodiments, the epigenetic readers may comprise recombinant methyl-CpG binding proteins Mecp2, Mbd1-6, SETDB1, SETDB2, TIP5/BAZ2A, Zbtb38, Kaiso, Zbtb4, Np95, Np97, DnaA, SeqA, MutHLS, Lrp, OxyR, Fur, HdfR or the recombinant methyl-binding domains derived therefrom. In some embodiments, the epigenetic readers may comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. In some embodiments, the epigenetic readers may comprise the microbial proteins Dam, CcrM, ModA13, SpnD39III, Dem, JHP1050, M2.Hpy.AII, or the recombinant methyl-binding domains derived therefrom. In some embodiments, the epigenetic writers and erasers may be catalytically inactive. In some embodiments, the epigenetic readers, writers, and erasers may comprise an epitope tag. In some embodiments, the epitope tag may comprise a N- or C-terminal 6x-histidine tag, green fluorescent protein (MA), myc, hemagglutinin (HA), Fc fusion, molecular recognition motif or any combination thereof. In some embodiments, the molecular recognition motif may comprise a birA or sortase motif. In some embodiments, nucleic acid compositions may be concentrated the by a solid support, wherein the solid support may comprise covalently bonded complementary antibodies to the epitope tag. In some embodiments, the specific affinity reagents may comprise a region to recognize and bind to the epigenetic feature. In some embodiments, the affinity targeting may comprise incubating the nucleic acid sample with a solid support comprising a plurality of immobilized affinity agents. In some embodiments, the plurality of immobilized affinity agents may comprise a region that may bind to the epigenetic feature. In some embodiments, the solid support may comprise a magnetic bead, an agarose bead, non-magnetic latex, functionalized Sepharose, pH-sensitive polymers or any combinations thereof. In some embodiments, the genome database may be a human genome database.

In some embodiments, the mammalian feature abundance tables may comprise mammalian genomic coordinates or annotated genomic loci and the number of sequencing reads associated therewith. In some embodiments, the mammalian feature abundance tables may comprise mammalian functional gene and biochemical pathway abundance tables. In some embodiments, the non-mammalian feature abundance tables may comprise non-mammalian taxonomic assignments and the number of sequencing reads associated therewith. In some embodiments, the non-mammalian feature abundance tables may comprise non-mammalian functional gene and biochemical pathway abundance tables. In some embodiments, the output of the trained predictive model may comprise an analysis of the combined mammalian and non-mammalian feature sets. In some embodiments, the trained predictive model may be trained with a set of mammalian and non-mammalian epigenomic abundances that are known to be present with a characteristic abundance or absent in a disease of interest. In some embodiments, the diagnostic model may utilize epigenomic abundance information from one or more of the following kingdoms of life: mammalian, bacterial, archaeal, fungal, and/or viral. In some embodiments, the diagnostic model may diagnose a category or tissue-specific location of disease. In some embodiments, the diagnostic model may be used to diagnose one or more types of cancer in a subject. In some embodiments, the diagnostic model may be used to diagnose one or more subtypes of cancer in a subject. In some embodiments, the diagnostic model may be used to predict the stage of cancer in a subject and/or predict cancer prognosis in the subject. In some embodiments, the diagnostic model may be used to predict cancer therapy response of the subject. In some embodiments, the diagnostic model may be utilized to select an optimal therapy for a particular subject. In some embodiments, the diagnostic model may be utilized to longitudinally model a course of one or more cancers' response to a therapy and to then adjust a treatment regimen.

In some embodiments, the diagnostic model may diagnose one or more of the following: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum cell tumors, thymoma, thyroid carcinoma, uterine carcinosarcoma, uterine corpus endometrial carcinoma, or uveal melanoma. In some embodiments, the diagnostic model may identify and remove certain non-human features as contaminants termed noise, while selectively retaining other non-human features termed signal. In some embodiments, the diagnostic model may be used to diagnose systemic lupus erythematosus, type 2 diabetes, chronic obstructive pulmonary disease (COPD), or sarcoidosis. In some embodiments, the liquid biopsy sample may include but is not limited to one or more of the following: plasma, serum, whole blood, urine, cerebral spinal fluid, saliva, sweat, tears, or exhaled breath condensate.

Aspects of the disclosure provided herein comprise a method of creating a feature set for a disease of one or more subjects, the method comprising: (a) providing one or more mammalian and non-mammalian nucleic acid molecules of a biological sample of one or more subjects with a disease: (b) enriching the one or more mammalian and non-mammalian nucleic of the biological sample of the one or more subjects by affinity targeting of an epigenetic feature common to the one or more mammalian and non-mammalian nucleic acid molecules; (c) sequencing the enriched one or more mammalian and non-mammalian nucleic acid molecules to generate one or more mammalian and non-mammalian sequencing reads; (d) filtering the mammalian and non-mammalian sequencing reads to isolate the non-mammalian sequencing reads thereby producing a mammalian features abundance; (e) analyzing the non-mammalian sequencing reads to generate a non-mammalian features abundance; and (f) creating the feature set by combining the mammalian and non-mammalian features abundance and the disease of the one or more subjects. In some embodiments, the epigenetic feature comprises a nucleic acid epigenetic feature. In some embodiments, the biological sample comprises a tissue, liquid biopsy sample or any combination thereof. In some embodiments, the one or more subjects are human or a non-human mammal. In some embodiments, the mammalian and non-mammalian nucleic acid molecules comprise DNA, RNA, cell-free DNA, cell-free RNA, exosomal DNA, exosomal RNA, or any combination thereof. In some embodiments, the affinity targeting of the nucleic acid epigenetic feature comprises concentrating the nucleic acid epigenetic feature. In some embodiments, the shared nucleic acid epigenetic feature comprises methylated CpG dinucleotides pairs, unmethylated CpG dinucleotide pairs, or any combination thereof. In some embodiments, the nucleic acid epigenetic feature comprises nucleobases 5-methylcytosine, 5-hydroxymethylcytosine, N4-acetylcytosine, N6-methyladenine, or any combination thereof.

In some embodiments, the affinity targeting utilizes specific affinity reagents to bind to the epigenetic feature. In some embodiments, the specific affinity reagents comprise streptavidin, NeutrAvidin, polyclonal, monoclonal, recombinant antibodies, aptamers, recombinant epigenetic proteins, or any combination thereof. In some embodiments, the recombinant epigenetic proteins comprise epigenetic readers, writers, erasers, or any combination thereof. In some embodiments, the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom. In some embodiments, the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. In some embodiments, the epigenetic writers and erasers are catalytically inactive. In some embodiments, the epigenetic readers, writers, and erasers comprise an epitope tag. In some embodiments, the epitope tag comprises a N- or C-terminal 6x-histidine tag, green fluorescent protein (MA), myc, hemagglutinin (HA), Fc fusion, molecular recognition motif, or any combination thereof. In some embodiments, the molecular recognition motif comprises a birA or sortase motif. In some embodiments, the method further comprises concentrating the mammalian and non-mammalian nucleic acid molecules by a solid support, wherein the solid support comprises immobilized complementary antibodies to the epitope tag. In some embodiments, the immobilized complementary antibodies are immobilized to the solid support by passive, electrostatic, covalently, or any combination thereof forces. In some embodiments, the specific affinity reagents comprise a region to recognize and bind to the epigenetic feature. In some embodiments, the affinity targeting comprises incubating the mammalian and non-mammalian nucleic acid molecules with a solid support comprising a plurality of immobilized affinity agents. In some embodiments, the plurality of immobilized affinity agents is immobilized to the solid support by passive, electrostatic, covalently, or any combination thereof forces.

In some embodiments, the plurality of immobilized affinity agents comprises a region that will bind to the epigenetic feature. In some embodiments, the solid support comprises a magnetic bead, an agarose bead, non-magnetic latex, functionalized Sepharose, pH-sensitive polymers, or any combinations thereof.

In some embodiments, the filtering comprises filtering the mammalian and non-mammalian sequencing reads against a genome database. In some embodiments, the genome database is a human genome database.

In some embodiments, the mammalian features abundance comprises mammalian genomic coordinates or annotated genomic loci and a number of sequencing reads associated therewith. In some embodiments, the mammalian features abundance comprises mammalian functional gene and biochemical pathway abundance tables. In some embodiments, the non-mammalian features abundance comprise non-mammalian taxonomic assignments and a number of sequencing reads associated therewith. In some embodiments, the non-mammalian features abundance comprises non-mammalian functional gene and biochemical pathway abundance tables. In some embodiments, the liquid biopsy sample includes but is not limited to one or more of the following: plasma, serum, whole blood, urine, cerebral spinal fluid, saliva, sweat, tears, or exhaled breath condensate.

Aspects of the disclosure provided herein, in some embodiments, comprise a method of using an output of a predictive model for determining a disease of a subject, the method comprising: (a) enriching one or more mammalian and non-mammalian nucleic acid molecules of a biological sample of a first set of subjects with a first disease and a second set of subjects with a second disease by affinity targeting of an epigenetic feature common to the one or more mammalian and non-mammalian nucleic acid molecules of the first and the second set of subjects; (b) sequencing the enriched one or more mammalian and non-mammalian nucleic acid molecules of the first and second subjects to generate one or more mammalian and non-mammalian sequencing reads; (c) filtering the first and second set of mammalian and non-mammalian sequencing reads to isolate the non-mammalian sequencing reads thereby producing a first and second set of mammalian features abundance; (d) analyzing the first and second set of non-mammalian sequencing reads to generate a first and second set of non-mammalian features abundance; (e) training a predictive model with the first set of mammalian and non-mammalian features abundance and the first disease of the first set of subjects thereby producing a trained predictive model; (f) and using the second set of mammalian and non-mammalian features abundance as an input to the trained predictive model to receive an output of the second disease of the second set of subjects. In some embodiments, the first or second set of subjects comprise one or more subjects. In some embodiments, the genome database is a human genome database. In some embodiments, the non-mammalian nucleic acid molecules comprise non-mammalian nucleic acid molecules. In some embodiments, the biological sample is derived from a tissue, liquid biopsy sample, or any combination thereof. In some embodiments, the first or second set of subjects are human or a non-human mammal. In some embodiments, the first or second set of mammalian and non-mammalian nucleic acid molecules comprise a total population of DNA, RNA, cell-free DNA, cell-free RNA, exosomal DNA, exosomal RNA, or any combination thereof. In some embodiments, the affinity targeting comprises concentrating the first and second set of mammalian and non-mammalian nucleic acid epigenetic features.

In some embodiments, the first and the second set of mammalian nucleic acid epigenetic features comprise the modified nucleobases 5-methylcytosine, 5-hydroxymethylcytosine, 5-formylcytosine, 5-carboxycytosine, N4-acetylcytosine, and N6-methyladenine. In some embodiments, the first and second set of non-mammalian nucleic acid epigenetic features comprise the modified nucleobases 5-methylcytosine, 5-hydroxymethylcytosine, 4-methylcytosine, N4-acetylcytosine, N6-methyladenine. In some embodiments, the first and second set of non-mammalian nucleic acid epigenetic feature comprises phosphorothioate-linked nucleotides. In some embodiments, the affinity targeting comprises specific affinity reagents. In some embodiments, the specific affinity reagents comprise streptavidin, NeutrAvidin, polyclonal, monoclonal, recombinant antibodies, aptamers, or recombinant epigenetic proteins.

In some embodiments, the recombinant epigenetic proteins comprise epigenetic readers, writers, erasers, or any combination thereof. In some embodiments, the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97, DnaA, SeqA, MutHLS, Lrp, OxyR, Fur, HdfR or the recombinant methyl-binding domains derived therefrom. In some embodiments, the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. In some embodiments, the epigenetic readers comprise the microbial proteins Dam, CcrM, ModA13, SpnD39III, Dem, JHP1050, M2.Hpy.AII, or the recombinant methyl-binding domains derived therefrom. In some embodiments, the epigenetic writers and erasers are catalytically inactive. In some embodiments, the epigenetic readers, writers, and erasers comprise an epitope tag. In some embodiments, the epitope tag comprises a N- or C-terminal 6x-histidine tag, green fluorescent protein (MA), myc, hemagglutinin (HA), Fc fusion, molecular recognition motif or any combination thereof. In some embodiments, the molecular recognition motif comprises a birA or sortase motif.

In some embodiments, the method further comprising concentrating the first or second mammalian or non-mammalian nucleic acid molecules by a solid support, wherein the solid support comprises immobilized complementary antibodies to the epitope tag. In some embodiments, the complementary antibodies are immobilized to the solid support by passive, electrostatic, covalently, or any combination thereof forces. In some embodiments, the specific affinity reagents comprise a region to recognize and bind to the first or second mammalian or non-mammalian epigenetic features. In some embodiments, the affinity targeting comprises incubating the first or second set of mammalian or non-mammalian nucleic acid molecules with a solid support comprising a plurality of immobilized affinity agents. In some embodiments, the affinity agents are immobilized by electrostatic, passive, covalent, or any combination thereof forces. In some embodiments, the plurality of immobilized affinity agents comprises a region that will bind to the first or second set of mammalian or non-mammalian epigenetic features. In some embodiments, the solid support comprises a magnetic bead, an agarose bead, non-magnetic latex, functionalized Sepharose, pH-sensitive polymers or any combinations thereof.

In some embodiments, the first or second set of mammalian features abundance comprise mammalian genomic coordinates or annotated genomic loci and a number of sequencing reads associated therewith. In some embodiments, the first or second set of mammalian features abundance comprise mammalian functional gene and biochemical pathway abundance tables. In some embodiments, the first or second set of non-mammalian features abundance comprise non-mammalian taxonomic assignments and a number of sequencing reads associated therewith. In some embodiments, the non-mammalian features abundance comprises non-mammalian functional gene and biochemical pathway abundance tables. In some embodiments, the output of the trained predictive model comprises an analysis of the combined first and second set of mammalian and non-mammalian features abundance. In some embodiments, an input of the trained predictive model comprises epigenomic abundance information from one or more of the following kingdoms of life: mammalian, bacterial, archaeal, fungal, and/or viral. In some embodiments, the first or second disease comprise a category or tissue-specific location of disease. In some embodiments, the first or second disease further comprise one or more types of cancer, one or more subtypes of cancer, stage of cancer, cancer prognosis, or any combination thereof.

In some embodiments, the trained predictive model is used to predict cancer therapy response of the second set of subjects. In some embodiments, the trained predictive model is utilized to select an optimal therapy for the second set of subjects. In some embodiments, the trained predictive model is utilized to longitudinally model a course of one or more cancers of the second set of subjects response to a therapy and to then adjust a treatment regimen.

In some embodiments, the first or second disease further comprises one or more of the following: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, or uveal melanoma.

In some embodiments, the predictive model is configured to remove contaminate non-mammalian features while selectively retaining other non-contaminate non-mammalian features. In some embodiments, the first or second disease further comprise lupus erythematosus, type 2 diabetes, chronic obstructive pulmonary disease (COPD), sarcoidosis, or any combination thereof. In some embodiments, the liquid biopsy sample comprises one or more of the following: plasma, serum, whole blood, urine, cerebral spinal fluid, saliva, sweat, tears, or exhaled breath condensate.

Aspects of the disclosure provided herein, in some embodiments, comprise a method of determining a disease of a subject, comprising: providing a biological sample of a subject; enriching one or more nucleic acid molecules of the biological sample by affinity targeting of an epigenetic feature common to the one or more nucleic acid molecules; sequencing the enriched one or more nucleic acid molecules to generate one or more nucleic acid molecule sequencing reads; and determining the disease of the subject as an output of a predictive model when the predictive model is provided the enriched one or more nucleic acid molecules as an input. In some embodiments, the one or more nucleic acid molecules comprise one or more mammalian nucleic acid molecules, one or more non-mammalian nucleic acid molecules, or a combination thereof. In some embodiments, the disease comprises cancer or a non-cancerous disease. In some embodiments, the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the non-cancerous disease comprises lupus erythematosus, type 2 diabetes, chronic obstructive pulmonary disease (COPD), sarcoidosis, or any combination thereof non-cancer diseases. In some embodiments, the method further comprises filtering the one or more nucleic acid molecules sequencing reads to identify one or more non-mammalian sequencing reads and one or more mammalian sequencing reads. In some embodiments, the epigenetic feature comprises a nucleic acid epigenetic feature. In some embodiments, the epigenetic feature comprises a mammalian nucleic acid epigenetic feature or a non-mammalian nucleic acid epigenetic feature. In some embodiments, the non-mammalian nucleic acid epigenetic feature comprises phosphorothioate-linked nucleotides. In some embodiments, the biological sample comprises a tissue, liquid biopsy sample or a combination thereof. In some embodiments, the subject is human or a non-human mammal. In some embodiments, the one or more mammalian nucleic acid molecules, comprise DNA, RNA, cell-free DNA, cell-free RNA, exosomal DNA, exosomal RNA, or any combination thereof. In some embodiments, the one or more non-mammalian nucleic acid molecules, comprise DNA, RNA, cell-free DNA, cell-free RNA, exosomal DNA, exosomal RNA, or any combination thereof. In some embodiments, the affinity targeting of the nucleic acid epigenetic feature comprises concentrating the nucleic acid epigenetic feature. In some embodiments, the nucleic acid epigenetic feature comprises methylated CpG dinucleotides pairs, unmethylated CpG dinucleotide pairs, or a combination thereof. In some embodiments, the nucleic acid epigenetic feature comprises nucleobases 5-methylcytosine, 5-hydroxymethylcytosine, N4-acetylcytosine, N6-methyladenine, or any combination thereof. In some embodiments, the affinity targeting utilizes specific affinity reagents to bind to the epigenetic feature. In some embodiments, the specific affinity reagents comprise streptavidin. NeutrAvidin, polyclonal, monoclonal, recombinant antibodies, aptamers, recombinant epigenetic proteins, or any combination thereof. In some embodiments, the recombinant epigenetic proteins comprise epigenetic readers, writers, erasers, or any combination thereof. In some embodiments, the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom. In some embodiments, the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. In some embodiments, the epigenetic readers comprise the microbial proteins Dam, CcrM, ModA13, SpnD39III, Dcm, JHP1050, M2.Hpy.AII, or the recombinant methyl-binding domains derived therefrom. In some embodiments, the epigenetic writers and erasers are catalytically inactive. In some embodiments, the epigenetic readers, writers, and erasers comprise an epitope tag. In some embodiments, the epitope tag comprises a N- or C-terminal 6x-histidine tag, green fluorescent protein (MA), myc, hemagglutinin (HA), Fc fusion, molecular recognition motif, or any combination thereof. In some embodiments, the molecular recognition motif comprises a birA or sortase motif. In some embodiments, the method further comprises concentrating the one or more mammalian and non-mammalian nucleic acid molecules by a solid support, wherein the solid support comprises immobilized complementary antibodies to the epitope tag. In some embodiments, the specific affinity reagents comprise a region to recognize and bind to the epigenetic feature. In some embodiments, affinity targeting comprises incubating the biological sample with a solid support comprising a plurality of immobilized affinity agents. In some embodiments, the plurality of immobilized affinity agents comprises a region that will bind to the epigenetic feature. In some embodiments, the solid support comprises a magnetic bead, an agarose bead, non-magnetic latex, functionalized Sepharose, pH-sensitive polymers, or any combination thereof. In some embodiments, filtering comprises filtering the one or more mammalian sequencing reads and the one or more non-mammalian sequencing reads against a genome database. In some embodiments, the genome database is a human genome database. In some embodiments, the predictive model is trained with one or more mammalian, one or more non-mammalian, or a combination thereof features determined from one or more subjects' one or more nucleic acid molecules of a biological sample and a corresponding disease of the one or more subjects. In some embodiments, the one or more mammalian feature comprises mammalian genomic coordinates or annotated genomic loci and a number of sequencing reads associated therewith. In some embodiments, the one or more mammalian feature comprises mammalian functional gene and biochemical pathway abundances. In some embodiments, the one or more non-mammalian feature comprises microbial taxonomic assignments and a number of sequencing reads associated therewith. In some embodiments, the one or more non-mammalian features comprises microbial functional gene and biochemical pathway abundances. In some embodiments, the liquid biopsy sample comprises plasma, serum, whole blood, urine, cerebral spinal fluid, saliva, sweat, tears, exhaled breath condensate, or any combination thereof. In some embodiments, the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when the one or more nucleic acid molecules of the biological sample are not enriched. In some embodiments, the predictive model comprises an area under the curve of at least about 0.70, at least about 0.80, at least about 0.85, at least about 0.90, or at least about 0.95 when determining the disease of the subject. In some embodiments, an output of the trained predictive model comprises an analysis of a combination of the one or more mammalian features and the one or more non-mammalian features abundance. In some embodiments, an input of the trained predictive model comprises epigenomic abundance information from one or more of the following kingdoms of life: mammalian, bacterial, archaeal, fungal, and/or viral. In some embodiments, the predictive model is further trained with a tissue-specific location of the disease. In some embodiments, the predictive model is further trained with the cancer's type, subtype, stage, prognosis, or any combination thereof. In some embodiments, the predictive model outputs the cancer's type, subtype, stage, prognosis, or any combination thereof when provided the subject's nucleic acid sequencing reads of the biological sample. In some embodiments, the predictive model outputs the subject's cancer therapy response. In some embodiments, the trained predictive model outputs a therapy for the subject that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the subject. In some embodiments, the trained predictive model outputs a longitudinal model of the subject's cancer in response to a therapy, an adjustment to a therapy to treat the subject's cancer, or a combination thereof. In some embodiments, the predictive model removes contaminate non-mammalian features while selectively retaining other non-contaminate non-mammalian features. In some embodiments, enriching reduces a total of the one or more nucleic acid molecules by at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99%.

Aspects of the disclosure provided herein, in some embodiments, comprise a method of training a predictive model, comprising: providing a biological sample of one or more subjects with a disease; enriching the biological sample of the one or more subjects by affinity targeting of an epigenetic feature common to one or more nucleic acid molecules of the biological sample; sequencing the enriched one or more nucleic acid molecules to generate one or more nucleic acid molecule sequencing reads; and training the predictive model with one or more features of the one or more nucleic acid molecule sequencing reads and the disease of the one or more subjects. In some embodiments, the epigenetic feature comprises a mammalian epigenetic feature or a non-mammalian epigenetic feature. In some embodiments, the one or more features comprise one or more disease features. In some embodiments, the trained predictive model determines a disease of another one or more subjects that differ from the one or more subjects when the trained predictive model is provided the another one or more subjects nucleic acid sequencing reads of a biological sample. In some embodiments, the one or more nucleic acid molecules comprise one or more mammalian nucleic acid molecules, one or more non-mammalian nucleic acid molecules, or a combination thereof. In some embodiments, the method further comprises filtering the one or more nucleic acid sequencing reads to identify one or more non-mammalian sequencing reads, the one or more mammalian sequencing reads, or a combination thereof. In some embodiments, the epigenetic feature comprises a nucleic acid epigenetic feature. In some embodiments, the biological sample comprises a tissue, liquid biopsy sample or a combination thereof. In some embodiments, the one or more subjects are human or a non-human mammal. In some embodiments, the one or more mammalian nucleic acid molecules comprise DNA, RNA, cell-free DNA, cell-free RNA, exosomal DNA, exosomal RNA, or any combination thereof. In some embodiments, the one or more non-mammalian nucleic acid molecules comprise DNA, RNA, cell-free DNA, cell-free RNA, exosomal DNA, exosomal RNA, or any combination thereof. In some embodiments, the affinity targeting of the nucleic acid epigenetic feature comprises concentrating the nucleic acid epigenetic feature. In some embodiments, the nucleic acid epigenetic feature comprises methylated CpG dinucleotides pairs, unmethylated CpG dinucleotide pairs, or a combination thereof. In some embodiments, the nucleic acid epigenetic feature comprises nucleobases 5-methylcytosine, 5-hydroxymethylcytosine, N4-acetylcytosine, N6-methyladenine, or any combination thereof. In some embodiments, affinity targeting utilizes specific affinity reagents to bind to the epigenetic feature. In some embodiments, the specific affinity reagents comprise streptavidin, NeutrAvidin, polyclonal, monoclonal, recombinant antibodies, aptamers, recombinant epigenetic proteins, or any combination thereof. In some embodiments, the recombinant epigenetic proteins comprise epigenetic readers, writers, erasers, or any combination thereof. In some embodiments, the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom. In some embodiments, the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. In some embodiments, the epigenetic writers and erasers are catalytically inactive. In some embodiments, the epigenetic readers, writers, and erasers comprise an epitope tag. In some embodiments, the epitope tag comprises a N- or C-terminal 6x-histidine tag, green fluorescent protein (MA), myc, hemagglutinin (HA), Fc fusion, molecular recognition motif, or any combination thereof. In some embodiments, the molecular recognition motif comprises a birA or sortase motif. In some embodiments, the method further comprises concentrating the one or more mammalian and the one or more non-mammalian nucleic acid molecules by a solid support, wherein the solid support comprises immobilized complementary antibodies to the epitope tag. In some embodiments, the specific affinity reagents comprise a region to recognize and bind to the epigenetic feature. In some embodiments, affinity targeting comprises incubating the biological sample with a solid support comprising a plurality of immobilized affinity agents. In some embodiments, the plurality of immobilized affinity agents comprises a region that will bind to the epigenetic feature. In some embodiments, the solid support comprises a magnetic bead, an agarose bead, non-magnetic latex, functionalized Sepharose, pH-sensitive polymers, or any combination thereof. In some embodiments, filtering comprises filtering the one or more mammalian and non-mammalian sequencing reads against a genome database. In some embodiments, the genome database is a human genome database. In some embodiments, the one or more features comprise one or more mammalian features, one or more non-mammalian features, or a combination thereof features. In some embodiments, the one or more mammalian features comprise mammalian genomic coordinates or annotated genomic loci and a number of sequencing reads associated therewith. In some embodiments, the one or more mammalian features comprise mammalian functional gene and biochemical pathway abundances. In some embodiments, the one or more non-mammalian features comprise microbial taxonomic assignments and a number of sequencing reads associated therewith. In some embodiments, the one or more non-mammalian features comprises microbial functional gene and biochemical pathway abundances. In some embodiments, the liquid biopsy sample comprises plasma, serum, whole blood, urine, cerebral spinal fluid, saliva, sweat, tears, exhaled breath condensate, or any combination thereof. In some embodiments, the disease comprises cancer or non-cancerous disease. In some embodiments, the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when one or more nucleic acid molecules of the biological sample are not enriched. In some embodiments, the predictive model comprises an area under the curve of at least about 0.70, at least about 0.80, at least about 0.85, at least about 0.90, or at least about 0.95 when determining the disease of the another one or more subjects. In some embodiments, the non-mammalian nucleic acid epigenetic feature comprises phosphorothioate-linked nucleotides. In some embodiments, the epigenetic readers comprise the microbial proteins Dam, CcrM, ModA13, SpnD39III, Dcm, JHP1050, M2.Hpy.AII, or the recombinant methyl-binding domains derived therefrom. In some embodiments, an output of the trained predictive model comprises an analysis of a combination of the one or more mammalian features and the one or more non-mammalian features abundance In some embodiments, an input of the trained predictive model comprises epigenomic abundance information from one or more of the following kingdoms of life: mammalian, bacterial, archaeal, fungal, and/or viral. In some embodiments, the predictive model is further trained with a tissue-specific location of the disease. In some embodiments, the predictive model is further trained with the cancer's type, subtype, stage, prognosis, or any combination thereof. In some embodiments, the predictive model outputs the cancer's type, subtype, stage, prognosis, or any combination thereof when provided the another one or more subjects' nucleic acid sequencing reads of the biological sample. In some embodiments, the trained predictive model outputs the another one or more subjects' cancer therapy response. In some embodiments, the trained predictive model outputs a therapy for the another one or more subjects that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the another one or more subjects. In some embodiments, the trained predictive model outputs a longitudinal model of the another one or more subjects' cancers in response to a therapy, an adjustment to a therapy to treat the subject's cancer, or a combination thereof. In some embodiments, the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the predictive model is configured to remove contaminate non-mammalian features while selectively retaining other non-contaminate non-mammalian features. In some embodiments, the non-cancerous disease comprises lupus erythematosus, type 2 diabetes, chronic obstructive pulmonary disease (COPD), sarcoidosis, or any combination thereof non-cancer diseases. In some embodiments, enriching reduces a total of the one or more nucleic acid molecules by at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99%.

Aspects of the disclosure provided herein, in some embodiments, comprise a computer system to determine a disease of a subject, comprising: one or more processors; and a non-transient computer readable storage medium including software, wherein the software comprises executable instruction that, as a result of execution, cause the one or more processors of the computer system to: (i) receive a subject's one or more nucleic acid molecule sequencing reads of one or more nucleic acid molecules of a biological sample, wherein the one or more nucleic acid molecules are enriched by affinity targeting of an epigenetic feature common to the one or more nucleic acid molecules; and (ii) determine a disease of the subject as an output of a predictive model when the predictive model is provided the one or more nucleic acid molecule sequencing. In some embodiments, the one or more nucleic acid molecules comprise one or more mammalian nucleic acid molecules, one or more non-mammalian nucleic acid molecules, or a combination thereof. In some embodiments, the disease comprises cancer or a non-cancerous disease. In some embodiments, the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the non-cancerous disease comprises lupus erythematosus, type 2 diabetes, chronic obstructive pulmonary disease (COPD), sarcoidosis, or any combination thereof non-cancer diseases. In some embodiments, the executable instruction further comprise filter the one or more nucleic acid molecules sequencing reads to identify one or more non-mammalian sequencing reads and one or more mammalian sequencing reads. In some embodiments, the epigenetic feature comprises a nucleic acid epigenetic feature. In some embodiments, the epigenetic feature comprises a mammalian nucleic acid epigenetic feature or a non-mammalian nucleic acid epigenetic feature. In some embodiments, the non-mammalian nucleic acid epigenetic feature comprises phosphorothioate-linked nucleotides. In some embodiments, the biological sample comprises a tissue, liquid biopsy sample or a combination thereof. In some embodiments, the subject is human or a non-human mammal. In some embodiments, the one or more mammalian nucleic acid molecules, comprise DNA. RNA, cell-free DNA, cell-free RNA, exosomal DNA, exosomal RNA, or any combination thereof. In some embodiments, the one or more non-mammalian nucleic acid molecules, comprise DNA, RNA, cell-free DNA, cell-free RNA, exosomal DNA, exosomal RNA, or any combination thereof. In some embodiments, the affinity targeting of the nucleic acid epigenetic feature comprises concentrating the nucleic acid epigenetic feature. In some embodiments, the nucleic acid epigenetic feature comprises methylated CpG dinucleotides pairs, unmethylated CpG dinucleotide pairs, or a combination thereof. In some embodiments, the nucleic acid epigenetic feature comprises nucleobases 5-methylcytosine, 5-hydroxymethylcytosine, N4-acetylcytosine, N6-methyladenine, or any combination thereof. In some embodiments, the affinity targeting utilizes specific affinity reagents to bind to the epigenetic feature. In some embodiments, the specific affinity reagents comprise streptavidin, NeutrAvidin, polyclonal, monoclonal, recombinant antibodies, aptamers, recombinant epigenetic proteins, or any combination thereof. In some embodiments, the recombinant epigenetic proteins comprise epigenetic readers, writers, erasers, or any combination thereof. In some embodiments, the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom. In some embodiments, the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. In some embodiments, the epigenetic readers comprise the microbial proteins Dam, CcrM, ModA13, SpnD39III, Dcm, JHP1050, M2.Hpy.AII, or the recombinant methyl-binding domains derived therefrom. In some embodiments, the epigenetic writers and erasers are catalytically inactive. In some embodiments, the epigenetic readers, writers, and erasers comprise an epitope tag. In some embodiments, the epitope tag comprises a N- or C-terminal 6x-histidine tag, green fluorescent protein (MA), myc, hemagglutinin (HA), Fc fusion, molecular recognition motif, or any combination thereof. In some embodiments, the molecular recognition motif comprises a birA or sortase motif. In some embodiments, the executable instructions further comprise concentrating the one or more mammalian and non-mammalian nucleic acid molecules by a solid support, wherein the solid support comprises immobilized complementary antibodies to the epitope tag. In some embodiments, the specific affinity reagents comprise a region to recognize and bind to the epigenetic feature. In some embodiments, the affinity targeting comprises incubating the biological sample with a solid support comprising a plurality of immobilized affinity agents. In some embodiments, the plurality of immobilized affinity agents comprises a region that will bind to the epigenetic feature. In some embodiments, the solid support comprises a magnetic bead, an agarose bead, non-magnetic latex, functionalized Sepharose, pH-sensitive polymers, or any combination thereof. In some embodiments, filtering comprises filtering the one or more mammalian sequencing reads and the one or more non-mammalian sequencing reads against a genome database. In some embodiments, the genome database is a human genome database. In some embodiments, the predictive model is trained with one or more mammalian, one or more non-mammalian, or a combination thereof features determined from one or more subjects' one or more nucleic acid molecules of a biological sample and a corresponding disease of the one or more subjects. In some embodiments, the one or more mammalian feature comprises mammalian genomic coordinates or annotated genomic loci and a number of sequencing reads associated therewith. In some embodiments, the one or more mammalian feature comprises mammalian functional gene and biochemical pathway abundances. In some embodiments, the one or more non-mammalian feature comprise microbial taxonomic assignments and a number of sequencing reads associated therewith. In some embodiments, the one or more non-mammalian features comprise microbial functional gene and biochemical pathway abundances. In some embodiments, the liquid biopsy sample comprises plasma, serum, whole blood, urine, cerebral spinal fluid, saliva, sweat, tears, exhaled breath condensate, or any combination thereof. In some embodiments, the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when the one or more nucleic acid molecules of the biological sample are not enriched. In some embodiments, the predictive model comprises an area under the curve of at least about 0.70, at least about 0.80, at least about 0.85, at least about 0.90, or at least about 0.95 when determining the disease of the subject. In some embodiments, an output of the trained predictive model comprises an analysis of a combination of the one or more mammalian features and the one or more non-mammalian features abundance. In some embodiments, an input of the trained predictive model comprises epigenomic abundance information from one or more of the following kingdoms of life: mammalian, bacterial, archaeal, fungal, and/or viral. In some embodiments, the predictive model is further trained with a tissue-specific location of the disease. In some embodiments, the predictive model is further trained with the cancer's type, subtype, stage, prognosis, or any combination thereof. In some embodiments, the predictive model outputs the cancer's type, subtype, stage, prognosis, or any combination thereof when provided the subject's nucleic acid sequencing reads of the biological sample. In some embodiments, the predictive model outputs the subject's cancer therapy response. In some embodiments, the trained predictive model outputs a therapy for the subject that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the subject. In some embodiments, the trained predictive model outputs a longitudinal model of the subject's cancer in response to a therapy, an adjustment to a therapy to treat the subject's cancer, or a combination thereof. In some embodiments, the predictive model removes contaminate non-mammalian features while selectively retaining other non-contaminate non-mammalian features. In some embodiments, the enriched nucleic acids comprise a reduction of at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99% of the one or more nucleic acid molecules prior to enrichment.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1F show flow diagrams of metaepigenomic workflows to produce a disease classification based on epigenetic features present within mammalian, bacterial, archaeal, fungal, and viral domains of life, as described in some embodiments herein.

FIGS. 2A-2E show exemplary mammalian nucleic acid modifications utilized for metaepigenomic analyses according to the methods of the present invention. FIG. 2A shows 5-methylcytosine (5mC). FIG. 2B shows 5-hydroxymethylcytosine (5hmC). FIG. 2C show 5-formylcytosine (5fC). FIG. 2D shows 5-carboxycytosine (5caC). FIG. 2E shows N4-acetycytosine (N4AcC), as described in some embodiments herein.

FIGS. 3A-3E show exemplary microbial nucleic acid modifications utilized for metaepigenomic analyses according to the methods of the present invention. FIG. 3A shows 6-methyladenine (6 mA). FIG. 3B shows 5-methylcytosine (5mC). FIG. 3C shows 4-methylcytosine (4mC). FIG. 3D shows N4-acetylcytosine (N4AcC). FIG. 3E shows 5-hydroxy methylcytosine (5hmC), as described in some embodiments herein.

FIG. 4 shows the bacterial and archaeal phosphorothioate modification utilized for metaepigenomic analyses according to the methods of the present invention, as described in some embodiments herein.

FIGS. 5A-5F show experimental data of microbial epigenetic biomarker discovery and cancer diagnostic model derived therefrom utilizing 5-hydroxymethylcytosine, an epigenetic feature ascribed hitherto as an exclusively mammalian epigenetic feature, as described in some embodiments, as described in some embodiments herein.

FIGS. 6A-6D show experimental data of microbial epigenetic biomarker discovery and cancer diagnostic model derived therefrom utilizing 5-hydroxymethylcytosine-based enrichment of microbial nucleic acids, as described in some embodiments herein.

FIG. 7 shows a diagram of a system configured to carry out, implement, and/or execute the methods described elsewhere herein, as described in some embodiments herein.

### DETAILED DESCRIPTION

Aspects of the disclosure provided herein may comprise a method of creating a diagnostic model for diagnosing disease in a subject based on the combination of mammalian and non-mammalian epigenetic information (herein denoted metaepigenomic' information, data, features, signatures, or biomarkers) contained in a nucleic acid sample. In some cases, the non-mammalian epigenetic information may comprise bacterial, fungal, archaeal, viral, or any combination thereof epigenetic information. This may be accomplished, in some embodiments, by identifying both mammalian and non-mammalian nucleic acid molecules isolated via antibody-based or non-antibody protein-based enrichment of genomic regions bearing one or more specific epigenetic marks and then testing the utility of those enriched nucleic acids for differentiating subjects with disease from those without. In some embodiments, the identified metaepigenomic biomarkers and their presence or abundance within a subject's sample can be used to assign a certain probability that (1) the individual has a specific disease; (2) the individual has a benign or malignant mass within a particular body site; (3) the individual has a particular type of benign or malignant mass; and/or (4) the disease has a high or low likelihood of responding to a particular therapy. Other uses for such methods are reasonably imaginable and readily implementable to those skilled in the art.

The invention disclosed herein, in some embodiments, may use metaepigenomic biomarkers derived from nucleic acids of mammalian and non-mammalian origin to diagnose a condition (i.e., cancer). In some embodiments, the disclosed invention may provide better clinical outcomes compared to a typical pathology report as it is not necessary to include one or more of observed tissue structure, cellular atypia, or other subjective measure traditionally used to diagnose cancer. In some embodiments, the disclosed method may provide a high degree of sensitivity by utilizing sequence information drawn from all possible genomes in sample rather than restricting analysis to the cancer genome, which is modified often at extremely low frequencies in a background of ‘normal’ human sources. In some embodiments, the methods disclosed herein may achieve such outcomes by either solid tissue or blood derived samples, the latter of which requires minimal sample preparation and is minimally invasive. In some embodiments, the liquid biopsy-based assay may overcome challenges posed by circulating tumor DNA (ctDNA) assays, which often suffer from sensitivity issues due to cell-free DNA (cfDNA) that originates from non-malignant human cells. In some embodiments, the liquid biopsy-based metaepigenomic assay may distinguish between cancer types, which ctDNA assays typically are not able to achieve, since most common cancer genomic aberrations are shared between cancer types (e.g., TP53 mutations, KRAS mutations). In some embodiments, the method described may constrain the size of the signatures, the method of which will be expected by someone knowledgeable in the art (e.g., regularized machine learning), the metaepigenomic assays may be made clinically available through the use of e.g., multiplexed quantitative polymerase chain reaction (qPCR), and targeted assay panels for multiplexed amplicon sequencing.

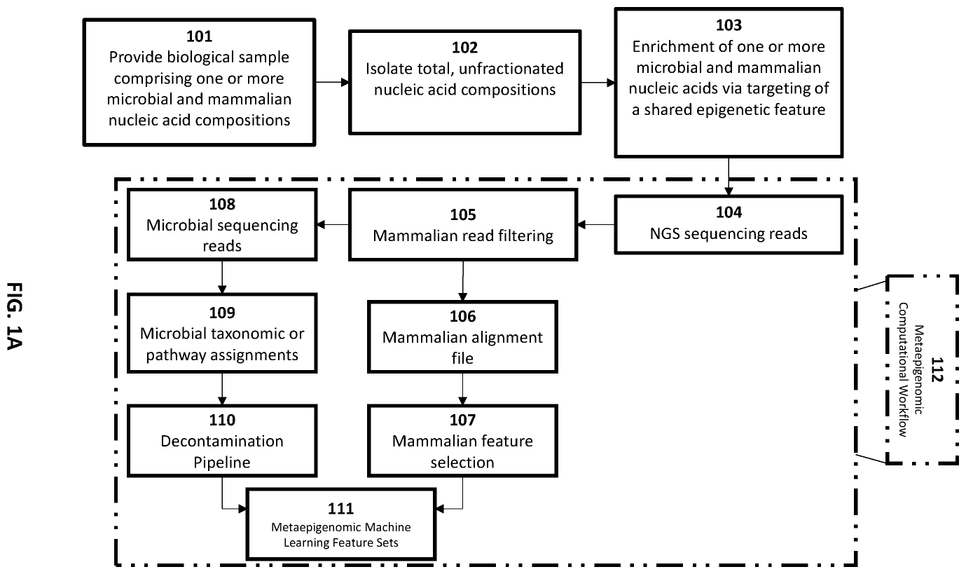

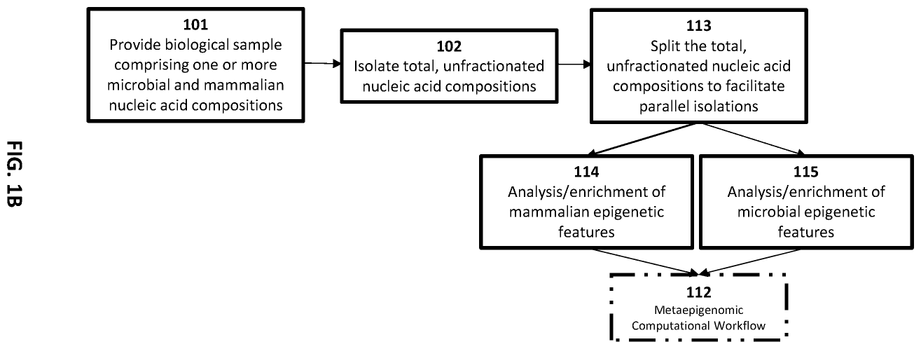

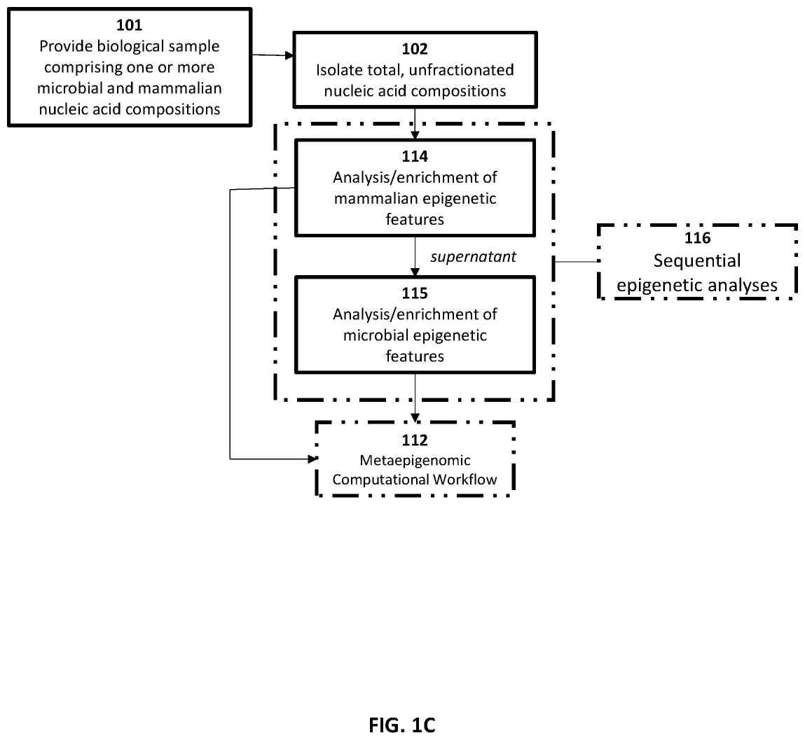

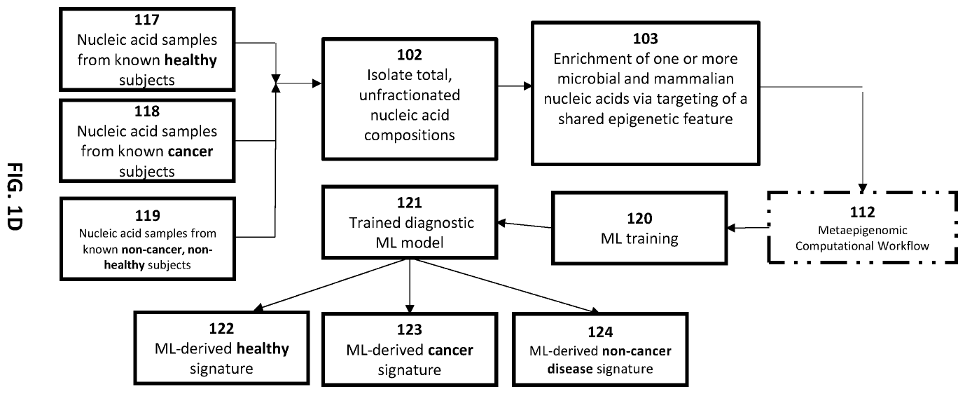

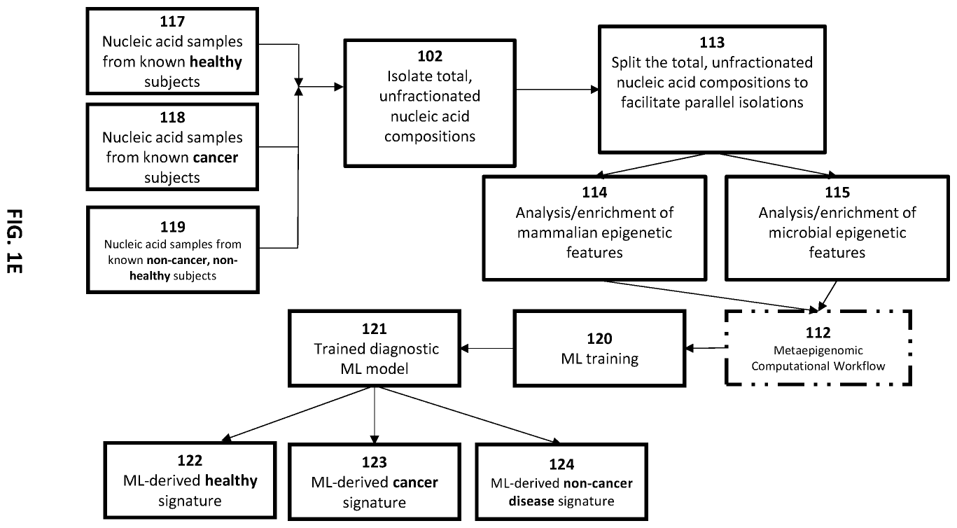

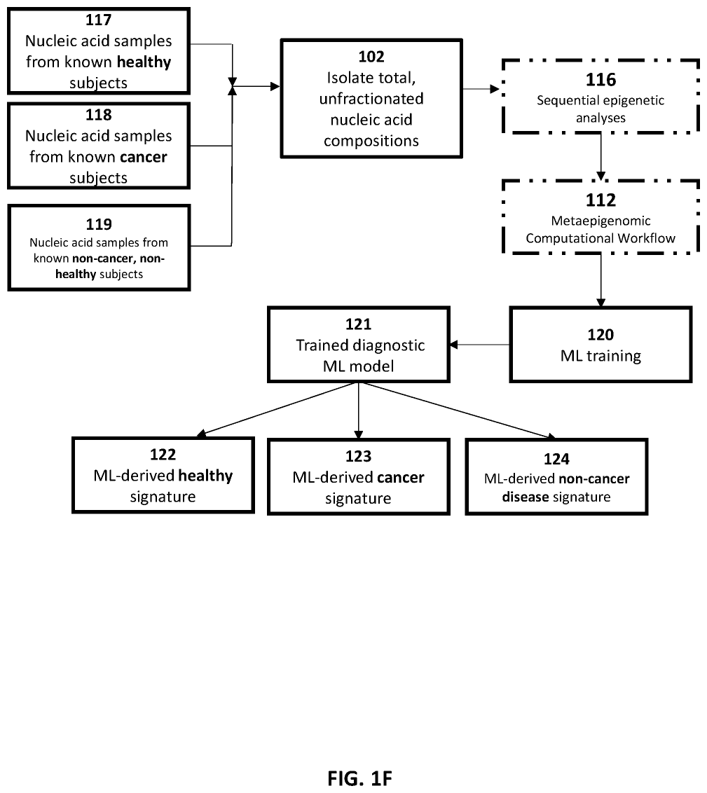

In some embodiments, the methods of the invention disclosed herein may comprise a method for creating a feature set for a disease of one or more subjects, as seen in FIG. 1A. In some cases, the method may comprise the steps of: (a) providing one or more mammalian and non-mammalian nucleic acid molecules of a biological sample of one or more subjects with a disease (e.g., cancer or non-cancerous disease) 101; (b) isolating total, unfractionated nucleic acid compositions 102; (c) enriching the one or more mammalian and non-mammalian nucleic acid molecules of the biological sample of the one or more subjects via targeting of a shared epigenetic feature 103; (d) sequencing the enriched one or more mammalian and non-mammalian nucleic acid molecules 104; (e) filtering the enriched one or more mammalian nucleic sequencing reads 105; (f) receiving from the result of the filtered one or more mammalian nucleic sequencing reads one or more non-mammalian sequencing reads 108; (g) generating taxonomic or pathways assignments for the one or more enriched non-mammalian sequencing reads, thereby generating non-mammalian feature abundances 109; (h) decontaminating the one or more non-mammalian feature abundances 110; (i) aligning the enriched one or more mammalian nucleic acid sequencing reads, thereby generating a mammalian alignment file 106; (j) selecting mammalian feature abundances of the one or more enriched mammalian nucleic acid sequencing reads of the mammalian alignment file 107; and (k) creating the feature set for the disease of the one or more subjects by combining the one or more mammalian and non-mammalian feature abundances and the disease of the one or more subjects into a feature set. In some cases, the feature set may comprise a metaepigenomic machine learning feature set 111. In some cases, the method may further comprise identifying disease-associated nucleic acid sequences of the mammalian or non-mammalian nucleic acid molecules bearing the epigenetic features in that dataset. In some cases, identification of the disease-associated nucleic acid sequences may originate from subjects with a disease (e.g., cancer or non-cancerous disease), or subjects that are healthy. In some instances, the diseased state may comprise cancer, diabetes, etc., or any disease or disorder discussed elsewhere herein. In some embodiments, the enriched sequencing data set may be acquired using next-generation sequencing, long-read sequencing (e.g., nanopore sequencing), or a combination thereof. In some embodiments, the enriched sequencing dataset 104 may result from affinity targeting of an epigenetic feature common to both mammalian and non-mammalian nucleic acid molecules with antibody or non-antibody protein-based agents specific for the shared epigenetic feature 103, thereby isolating genomic regions of interest from nucleic acid samples 102 from biological samples containing nucleic acid sequences of mammalian and non-mammalian origin 101 as shown in FIG. 1A. In some embodiments, the metaepigenomic features present in an enriched population of nucleic acids 103 may be identified through a metaepigenomic computational workflow 112 wherein enriched mammalian sequencing reads may be computationally filtered 105 from the total raw sequencing reads 104 via alignment to a mammalian reference genome using bowtie2 or Kraken or their equivalents to produce a mammalian alignment file. In some embodiments, the mammalian alignment file may be processed through an analysis pipeline 107 (such as MethylAction or MEDIPS) to identify genomic regions enriched via affinity targeting 103 of the select epigenetic feature, thereby producing an output of selected mammalian features. In some embodiments, the resulting non-mammalian reads 108 may be taxonomically classified using bowtie2 or Kraken with a reference microbial database, such as the Web of Life 109. In some embodiments, the abundance of non-mammalian genes bearing a specific epigenetic mark may be ascertained using the Web of Life Toolkit App (WolTka) or any equivalent thereof 109. In some embodiments, the identified non-mammalian reads 109 may be processed through a decontamination pipeline 110 to remove sequences derived from common non-mammalian contaminants to yield decontaminated non-mammalian features. In some embodiments, the decontaminated non-mammalian features 110 may be combined with the output of the mammalian analysis pipeline 107 to produce a metaepigenomic feature set 111 that may serve as training feature set for predictive models.

In some embodiments, the disclosure provided herein may comprise a method of preparing separate mammalian and non-mammalian epigenomic analysis through sample splitting and parallel isolation of nucleic acids based on different epigenetic features present in mammalian and non-mammalian domains, as seen in FIG. 1B. In some cases, the method may comprise the steps of: (a) providing a biological sample comprising one or more mammalian and non-mammalian nucleic acid compositions 101; (b) isolating unfractionated nucleic acid compositions 102; (c) dividing the isolated unfractionated nucleic acid compositions into one or more aliquots 113; (d) enriching mammalian and non-mammalian nucleic acid compositions of the one or more aliquots, thereby producing an enriched mammalian and non-mammalian nucleic acid compositions (114,155); and (e) converting the enriched mammalian and non-mammalian nucleic acid compositions to a feature set for a disease 112. In some cases, converting the enriched mammalian and non-mammalian nucleic acid molecule compositions to a feature set may comprise inputting the enriched sequencing reads into the metaepigenomic computational workflow 112 at the step of filtering the mammalian reads 105. In some cases, the sample of mammalian and non-mammalian nucleic acid molecules 102 may be physically split 113 to facilitate separate analyses of mammalian and non-mammalian (microbial) epigenetic features. In some embodiments, mammalian epigenetic features 114 may be enriched by affinity targeting of an epigenetic feature with antibody or non-antibody protein-based agents specific for the epigenetic feature. In some embodiments, the distribution of the epigenetic features throughout the mammalian genome may be ascertained by a specific sequencing method that may or may not utilize a first enrichment step such as bisulfite sequencing, reduced representation bisulfite sequencing, oxidative bisulfite sequencing. ACE-seq, enzymatic methyl-seq (EM-seq), nanopore sequencing or their equivalent. In some embodiments, non-mammalian epigenetic features 115 may be enriched by affinity targeting of an epigenetic feature with antibody or non-antibody protein-based agents specific for the epigenetic feature. In some embodiments, the distribution of the epigenetic feature throughout the non-mammalian genomes in a sample may be ascertained by a specific sequencing method that may or may not utilize a first enrichment step such as bisulfite sequencing, reduced representation bisulfite sequencing, oxidative bisulfite sequencing. ACE-seq, enzymatic methyl-seq (EM-seq), nanopore sequencing or their equivalent. In some embodiments, the results of the parallel mammalian 114 and non-mammalian 115 epigenetic analyses are combined and inputted into the metaepigenomic computational workflow 112 to yield metaepigenomic machine learning feature sets.

In some embodiments, the disclosure provided herein may comprise a method of generating a feature set of a disease of a subject through sequential isolation of mammalian and non-mammalian nucleic, as seen in FIG. 1C. In some cases, the method may comprise the steps of: (a) providing one or more biological samples of one or more subjects, wherein the biological samples comprise mammalian and non-mammalian nucleic acid compositions 101; (b) isolating unfractionated mammalian and non-mammalian nucleic acid compositions 102; (c) enriching the unfractionated mammalian and non-mammalian nucleic acid composition to separate mammalian nucleic acid compositions and a remainder composition 114; (d) enriching the remainder composition for non-mammalian nucleic acid compositions; (e) converting the mammalian and non-mammalian nucleic acid compositions into a feature set of a disease 112. In some cases, the converting of mammalian and non-mammalian nucleic acid compositions into a feature set of a disease may comprise inputting the mammalian and non-mammalian sequencing reads determined by 114 and 115 (FIG. 1C) into the metaepigenomic computational workflow 112 at element 104 (FIG. 1A). In some embodiments, the mammalian and non-mammalian epigenetic features may be enriched from the same nucleic acid sample 102 in sequential fashion 116 as shown in FIG. 1C, wherein mammalian epigenetic features 114 may be enriched by affinity targeting of an epigenetic feature with antibody or non-antibody protein-based agents specific for the epigenetic feature, thereby producing a sample depleted of mammalian nucleic acid molecules bearing the targeted epigenetic mark which sample may then serve as the input for non-mammalian epigenetic feature enrichment 115. In some embodiments, the order of enrichments is reversed, with targeted non-mammalian epigenetic enrichment 115 preceding mammalian epigenetic enrichment 114. The output of this sequential epigenetic analysis 116 may then be inputted into the metaepigenomic computational workflow 112 to yield metaepigenomic machine learning feature sets.

In some aspects, the disclosure provided herein may comprise a method of training a predictive model incorporating a metaepigenomic analysis module to enable metaepigenomic-based discovery of healthy, non-cancer (non-healthy) and cancer-associated non-mammalian signatures FIG. 1D. In some embodiments, the systems and methods of the invention disclosed herein may comprise (a) determining the metaepigenomic features of a sample via sequencing; and (b) generating a predictive model. In some embodiments, the sequencing method may comprise next-generation sequencing or long-read sequencing (e.g., nanopore sequencing) or a combination thereof. In some embodiments, the predictive model 121 may comprise a training a predictive model 120 on the metaepigenomic machine learning feature sets, described elsewhere herein. In some embodiments, the predictive model may be a regularized machine learning model. In some embodiments, the predictive model may comprise a linear regression, logistic regression, decision tree, support vector machine (SVM), naïve bayes, k-nearest neighbors (KNN), k-Means, random forest algorithm model or any combination thereof.

Aspects of the disclosure herein may comprise a method to train a predictive model to determine a disease of a subject, as seen in FIG. 1D. In some cases, the method may comprise the steps of: (a) providing one or more nucleic acid samples from healthy 117, cancerous 118, non-cancerous and non-healthy, or any combination thereof subjects; (b) isolating unfractionated nucleic acid compositions from the one or more nucleic acid samples 102; (c) enriching the one or more non-mammalian and mammalian nucleic acid molecules of the unfractionated nucleic acid composition by affinity targeting 103; (d) converting the one or more non-mammalian and mammalian nucleic acid molecules to one or more feature sets corresponding to a disease of the one or more subjects 112; and (e) train a predictive 120 model with the one or more feature sets and corresponding diseases, thereby producing a trained predictive model 121 configured to determine the disease of the subject. In some cases, the determined characterization of the subject may comprise healthy 122, cancerous 123, or non-cancerous disease 124. In some cases, the determined characterization of the subject may comprise healthy 122, cancerous 123, or non-cancerous disease 124. In some embodiments, the predictive model may be trained 120 with the metaepigenomic feature sets 112 derived from nucleic acids 102 from a plurality of known healthy subjects 117, a plurality of known cancer subjects 118, and a plurality of non-cancer, non-healthy subjects 119 that have been enriched by affinity targeting 103 of an epigenetic feature shared among the mammalian and non-mammalian nucleic acid molecules present in the samples as shown in FIG. 1D. In some embodiments, training of the predictive model 120 to produce a trained predictive model 121 yields machine learning-identified metaepigenomic signatures for healthy subjects 122, subjects with cancer 123, and non-healthy subjects without cancer 124.

Aspects of the disclosure provided herein may comprise a method of discrete mammalian and non-mammalian nucleic acid analysis to train a predictive model to determine a disease of a subject, as seen in FIG. 1E. In some cases, the method may comprise the steps of: (a) providing one or more nucleic acid samples from healthy 117, cancerous 118, non-cancerous and non-healthy, or any combination thereof subjects; (b) isolating unfractionated nucleic acid compositions from the one or more nucleic acid samples 102; (c) dividing the unfractionated nucleic acid composition 113 to 2 or more aliquots (114, 115); (d) enriching a first subset of the 2 or more aliquots for one or more mammalian nucleic 114 acid and a second subset of the 2 or more aliquots for one or more non-mammalian nucleic acid molecules 115; (e) converting the one or more non-mammalian and mammalian nucleic acid molecules to one or more feature sets corresponding to a disease of one or more subjects 112; and (e) train a predictive 120 model with the one or more feature sets and corresponding diseases, thereby producing a trained predictive model 121 configured to determine the disease of the subject. In some cases, the determined the disease of the subject may comprise healthy 122, cancerous 123, or non-cancerous disease 124. In some aspects, the disclosure provided herein may comprise a method of training a predictive model on metaepigenomic feature sets to enable metaepigenomic-based discovery of healthy, non-cancer (non-healthy) and cancer-associated non-mammalian signatures wherein the separate epigenetic analyses of FIG. 1B are joined to form a combined metaepigenomic feature set for predictive model training 120. In some embodiments, metaepigenomic feature sets 112 configured for training the predictive model 120 may be derived from nucleic acids 102 from a plurality of known healthy subjects 117, a plurality of known cancer subjects 118, and a plurality of non-cancer, non-healthy subjects 119 that have been physically split to facilitate parallel analyses of mammalian and non-mammalian epigenetic features as shown in FIG. 1E.

Aspects of the disclosure provided herein may comprise a method of sequential mammalian and non-mammalian nucleic acid analysis to train a predictive model to determine a disease of a subject. In some cases, the method may comprise the steps of: (a) providing one or more nucleic acid samples from healthy 117, cancerous 118, non-cancerous and non-healthy, or any combination thereof subjects; (b) isolating unfractionated nucleic acid compositions from the one or more nucleic acid samples 102; (c) conducting the sequential epigenetic analysis with the isolated unfractionated nucleic acid compositions, thereby producing one or more non-mammalian and mammalian nucleic acid molecules; (d) converting the one or more non-mammalian and mammalian nucleic acid molecules to one or more feature sets corresponding to a disease of one or more subjects 112; and (e) train a predictive 120 model with the one or more feature sets and corresponding diseases, thereby producing a trained predictive model 121 configured to determine the disease of the subject. In some cases, the sequential epigenetic analysis 116, as shown in FIG. 1C, comprises: enriching the unfractionated nucleic acid composition to separate mammalian nucleic acid compositions and a remainder composition 114; and enriching the remainder composition for non-mammalian nucleic acid compositions 115. In some cases, the determined characterization of the subject may comprise healthy 122, cancerous 123, or non-cancerous disease 124 In some aspects, the disclosure provided herein may comprise a method of training a predictive model to enable metaepigenomic-based discovery of healthy, non-cancer (non-healthy) and cancer-associated non-mammalian signatures wherein the sequential epigenetic analyses of FIG. 1C are joined to form a combined metaepigenomic feature set for machine learning. In some embodiments, metaepigenomic feature sets 112 to train the predictive model 120 may be derived from nucleic acids 102 from a plurality of known healthy subjects 117, a plurality of known cancer subjects 118, and a plurality of non-cancer, non-healthy subjects 119 that have undergone sequential analyses of mammalian and non-mammalian epigenetic features as shown in FIG. 1F.

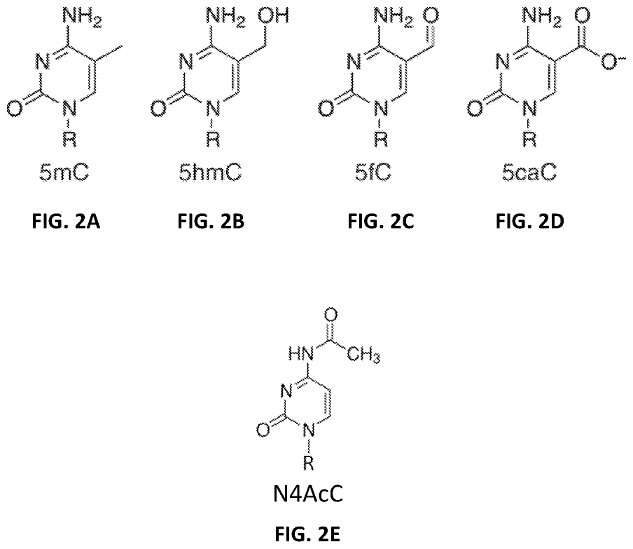

In some embodiments, the specific mammalian epigenetic features targeted for enrichment or direct sequencing analysis may comprise 5-methylcytosine (5mC), 5-hydroxymethylcytosine (5hmC), 5-formylcytosine (5fC), 5-carboxycytosine (5caC), or N4-acetylcytosine (N4AcC), as shown in FIG. 2 (FIG. 2A-2E, respectively).

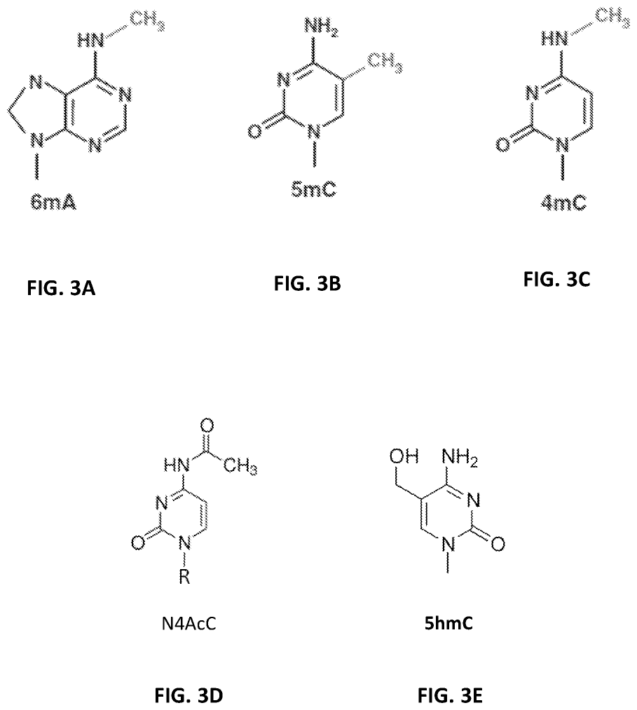

In some embodiments, the specific non-mammalian epigenetic features targeted for enrichment or direct sequencing analysis may comprise 6-methyladenosine (6 mA), 5-methylcytosine (5mC), 4-methylcytosine (4mC), N4-acetylcytosine (N4AcC), or 5-hydroxymethylcytosine (5hmC), as shown in FIG. 3 (FIG. 3A-3E, respectively).

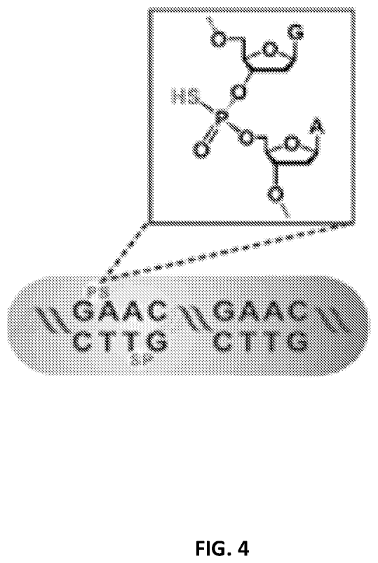

In some embodiments, the specific non-mammalian epigenetic feature targeted for enrichment may comprise the phosphorothioate nucleotide linkage shown in FIG. 4.

Aspects disclosed herein may provide a method of creating a predictive model for diagnosing disease in a subject based on the combination of mammalian and non-mammalian epigenetic information contained in nucleic acid samples (FIG. 1A) comprising: (a) enriching one or more mammalian and non-mammalian nucleic acid molecules by affinity targeting of epigenetic features present in the one or more mammalian and non-mammalian nucleic acid molecules 103; (b) sequencing the nucleic acids enriched through targeting of the epigenetic features 104; computationally analyzing 112 both the mammalian and non-mammalian sequencing reads from the dataset to produce metaepigenomic machine learning feature sets 111 that are used to train predictive models to produce a trained diagnostic model (FIG. 1D).

Aspects disclosed herein provide a method of training a predictive model (FIG. 1D) comprising: (a) providing as a training data set (i) one or more subjects' one or more sequenced metaepigenomic abundances 112; (b) providing as a test set (i) one or more subjects' one or more sequenced metaepigenomic abundances 112; (c) training the predictive model on a 60 to 40 sample ratio of training to validation samples, respectively; and (d) evaluating the predictive accuracy of the predictive model.

In some embodiments, the prediction made by the trained predictive model may comprise a machine learning signature indicative of a healthy subject, or a machine learning derived signature indicative of subject with cancer, or a machine learning derived signature indicative of a subject with a disease other than cancer. In some embodiments, the trained predictive model may identify and remove the one more non-mammalian or non-microbial nucleic acids classified as noise while selectively retaining other one or more non-mammalian or non-microbial sequences termed signal.

Although the above steps show each of the methods or sets of operations in accordance with embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial.

One or more of the steps of each of the methods or sets of operations may be performed with circuitry, for example, one or more of the processor or logic circuitry such as programmable array logic for a field programmable gate array. The circuitry may be programmed to provide one or more of the steps of each of the methods or sets of operations, and the program may comprise program instructions stored on a computer readable memory or programmed steps of the logic circuitry such as the programmable array logic or the field programmable gate array, for example.

### Predictive Models

The methods and systems of the present disclosure may utilize or access external capabilities of artificial intelligence, predictive models, and/or machine learning techniques to determine if one or more subjects have cancer from a biological sample of each subject of the one or more subjects. In some cases, the artificial intelligence techniques may identify features (e.g., non-mammalian and/or mammalian) of the one or more nucleic acid molecule sequencing reads that may predict a cancer of one or more subjects. In some cases, the features may be used to train one or more predictive models, described elsewhere herein. These features may be used to predict diseases or disorders with an accuracy, as described elsewhere herein. In some cases, the diseases or disorders may comprise cancer, or non-cancerous disease as described elsewhere herein. Using such a predictive model, algorithms and/or machine learning techniques, health care providers (e.g., physicians, nurses, medical technicians, etc.) may be able to make informed, accurate risk-based decisions, thereby improving early-stage disease diagnosis, disease progression and monitoring, treatment and/or therapeutic suggestions to treat a subject's disease, or any combination thereof.

The methods and systems of the present disclosure may analyze the presence and abundance of mammalian nucleic acid molecules and/or non-mammalian nucleic acid molecules to determine one or more mammalian features and/or one or more non-mammalian features that may predict a disease of one or more subjects. In some cases, the methods, and systems, described elsewhere herein, may train a predictive model with the one or more mammalian features, one or more non-mammalian features indicative of a disease, and a corresponding disease of one or more subject. In some cases, the trained predictive model may then be used to generate a likelihood (e.g., a prediction) of disease (e.g., cancer or non-cancerous diseases) of another one or more subjects that differ from the one or more subjects utilized to train the predictive model. The trained predictive model may comprise an artificial intelligence-based model, such as a machine learning based classifier, configured to process one or more nucleic acid molecule sequencing reads to generate the likelihood of a subject having the disease. The model may be trained using presence or abundance of one or more mammalian and/or non-mammalian nucleic acid sequencing reads generated from one or more nucleic acid molecules of a biological sample from one or more cohorts of patients, e.g., cancer patients, patients with non-cancerous diseases, patients with no disease and no cancer, cancer patients receiving a treatment for a cancer, patients receiving treatment for a non-cancerous disease, or any combination thereof. In some cases, the predictive model may be trained to provide a treatment prediction to treat a cancer of one or more patients that are not part of the training dataset of the predictive model. Such a predictive model may output a treatment recommendation for the one or more patients that are not part of the training dataset when provided an input of the patient's presence and abundance of one or more nucleic acid molecule sequencing reads obtain from a biologic sample.

The predictive model may comprise one or more predictive models. The predictive model may comprise one or more machine learning algorithms. Examples of machine learning algorithms may include a support vector machine (SVM), a naïve Bayes classification, a random forest, a neural network, a deep neural network (DNN), a recurrent neural network (RNN), a deep RNN, a long short-term memory (LSTM) recurrent neural network (RNN), a gated recurrent unit (GRU), a gradient boosting machine, linear regression, k-nearest neighbors, k-means, decision tree, logistic regression, other supervised learning algorithm or unsupervised machine learning model, or any combination thereof. The predictive model may be used for classification or regression. The model may involve the estimation of ensemble models, comprised of multiple predictive models, and utilize techniques such as gradient boosting, for example in the construction of gradient-boosting decision trees. The model may be trained using one or more training datasets corresponding to patient and/or subject data e.g., patient medical history, family medical history, blood pressure, pulse, temperature, oxygen saturation or any combination thereof in addition to one or more nucleic acid sequencing reads generated from one or more nucleic acid molecules of a subject's biological sample, described elsewhere herein.

Training datasets may be generated from, for example, one or more cohorts of patients having common clinical disease or disorder diagnosis. Training datasets may comprise a set of one or more non-mammalian features, one or more mammalian features, or a combination thereof in the form of presence and/or abundance of one or more mammalian nucleic acid molecules and/or one or more non-mammalian nucleic acid molecules of a biological sample of one or more subjects. In some instances, the one or more mammalian nucleic acid molecules and/or the one or more non-mammalian nucleic acid molecules may comprise enriched nucleic acid molecules, as described elsewhere herein. Features may comprise a corresponding cancer diagnosis of one or more subjects to aforementioned one or more mammalian and/or one or more non-mammalian features. In some cases, features may comprise patient information such as patient age, patient medical history, other medical conditions, current or past medications, clinical risk scores, and time since the last observation. For example, a set of features collected from a given patient at a given time point may collectively serve as a signature, which may be indicative of a disease or disease status of the patient and/or subject at a time point.

Labels of the training data may comprise clinical outcomes such as, for example, a presence, absence, diagnosis, or prognosis of a disease (e.g., cancer or non-cancerous disease) or disorder of the subject and/or patient. Clinical outcomes may comprise treatment efficacy (e.g., whether a subject is a positive responder to a cancer-based treatment).

Input features may be structured by aggregating the data into bins or alternatively using a one-hot encoding. Inputs may also include feature values or vectors derived from the previously mentioned inputs, such as cross-correlations.

Training records may be constructed from presence and/or abundance features of one or more mammalian nucleic acid molecules and/or one or more non-mammalian nucleic acid molecules of a biological sample of one or more subjects.

The model may process the input features to generate output values comprising one or more classifications, one or more predictions, or a combination thereof. For example, such classifications or predictions may include a binary classification of a cancer or no cancer present in a subject (e.g., absence of a disease or disorder), a classification between a group of categorical labels (e.g., ‘no disease or disorder’, ‘apparent disease or disorder”, and ‘likely disease or disorder), a likelihood (e.g., relative likelihood or probability) of developing a particular disease or disorder, a score indicative of a presence of disease or disorder, a ‘risk factor’ for the likelihood of mortality of the patient, and a confidence interval for any numeric predictions. Various machine learning techniques may be cascaded such that the output of a machine learning technique may also be used as input features to subsequent layers or subsections of a predictive model.

In order to train the model (e.g., by determining weights and correlations of the model) to generate real-time classifications or predictions, the model can be trained using datasets and/or features, described elsewhere herein. Such datasets may be sufficiently large to generate statistically significant classifications or predictions. For example, datasets may comprise: databases of data, where the data may comprise one or more nucleic acid molecule sequencing reads of one or more subjects and the corresponding disease label of the one or more subjects. The training data sets may be collected from training subjects (e.g., humans and/or non-human mammals). Each subject's training data set may have a diagnostic status indicating that the subject has been diagnosed with the disease (e.g., cancer or non-cancerous diseases) or have not been diagnosed with the biological condition.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. In some embodiments, leave one out cross validation may be employed. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

To improve the accuracy of predictive model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered or batch corrected to remove or mitigate confounding factors. For example, within a database, a subset of patients may be excluded.

The predictive model may comprise one or more neural networks, such as a neural network, a convolutional neural network (CNN), a deep neural network (DNN), a recurrent neural network (RNN), or a deep RNN. The recurrent neural network may comprise units which can be long short-term memory (LSTM) units or gated recurrent units (GRU). For example, the model may comprise an algorithm architecture comprising a neural network with a set of input features e.g., one or more nucleic acid molecule sequencing reads, vitals (as described elsewhere herein), patient medical history, and/or patient demographics. Neural network techniques, such as dropout or regularization, may be used during training of the predictive model to prevent overfitting. The neural network may comprise a plurality of sub-networks, each of which is configured to generate a classification or prediction of a different type of output information (e.g., which may be combined to form an overall output of the neural network). The machine learning model may alternatively utilize statistical or related algorithms including random forest, classification and regression trees, support vector machines, discriminant analyses, regression techniques, as well as ensemble and gradient-boosted variations thereof.

When the predictive model generates a classification or a prediction of a disease or disorder, a notification (e.g., alert or alarm) may be generated and transmitted to a health care provider, such as a physician, nurse, or other member of the patient's treatment team within a hospital and/or clinic. Notifications may be transmitted via an automated phone call, a short message service (SMS) or multimedia message service (MMS) message, an e-mail, or an alert within a dashboard. The notification may comprise output information such as a prediction of a disease or disorder, a likelihood of the predicted disease or disorder, a time until an expected onset of the disease or disorder, a confidence interval of the likelihood or time, or a recommended course of treatment for the disease or disorder.

To validate the performance of the predictive model, different performance metrics may be generated. For example, an area under the receiver-operating characteristic curve (AUROC) may be used to determine the diagnostic capability of the predictive model. For example, the predictive model may use classification thresholds which are adjustable, such that specificity and sensitivity are tunable, and the receiver-operating characteristic curve (ROC) can be used to identify the different operating points corresponding to different values of specificity and sensitivity.

In some cases, such as when datasets are not sufficiently large, cross-validation may be performed to assess the robustness of a model across different training and testing datasets.

To calculate performance metrics such as sensitivity, specificity, accuracy, positive predictive value (PPV), negative predictive value (NPV), area under the precision-recall curve (AUPR), AUROC, or similar, the following definitions may be used. A “false positive” may refer to an outcome in which a positive outcome or result has been incorrectly or prematurely generated (e.g., before the actual onset of, or without any onset of, the disease or disorder). A “true positive” may refer to an outcome in which positive outcome or result has been correctly generated, when the patient has the disease or disorder (e.g., the patient shows symptoms of the disease or disorder, or the patient's record indicates the disease or disorder). A “false negative” may refer to an outcome in which a negative outcome or result has been generated, but the patient has the disease or disorder (e.g., the patient shows symptoms of the disease or disorder, or the patient's record indicates the disease or disorder). A “true negative” may refer to an outcome in which a negative outcome or result has been generated (e.g., before the actual onset of, or without any onset of, the disease or disorder).

The predictive model may be trained until certain pre-determined conditions for accuracy or performance are satisfied, such as having minimum desired values corresponding to diagnostic accuracy measures. For example, the diagnostic accuracy measure may correspond to prediction of a likelihood of occurrence of a disease or disorder in the subject. As another example, the diagnostic accuracy measure may correspond to prediction of a likelihood of deterioration or recurrence of a disease or disorder for which the subject has previously been treated. Examples of diagnostic accuracy measures may include sensitivity, specificity, positive predictive value (PPV), negative predictive value (NPV), accuracy, AUPR, and AUROC corresponding to the diagnostic accuracy of detecting or predicting a disease or disorder.

For example, such a pre-determined condition may be that the sensitivity of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the disease or disorder comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPR) of predicting the disease or disorder comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the disease or disorder with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the model is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, independent component analysis (ICA) is used to de-dimensionalize the data, such as that described in Lee, T.-W. (1998): Independent component analysis: Theory and applications, Boston, Mass: Kluwer Academic Publishers, ISBN 0-7923-8261-7, and Hyvärinen, A.: Karhunen, J.: Oja, E. (2001): Independent Component Analysis, New York: Wiley, ISBN 978-0-471-40540-5, which is hereby incorporated by reference in its entirety.

In some embodiments, principal component analysis (PCA) is used to de-dimensionalize the data, such as that described in Jolliffe, I. T. (2002). Principal Component Analysis. Springer Series in Statistics. New York: Springer-Verlag. doi: 10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York: Mount, 2001, Bioinformatics: sequence and genome analysis. Cold Spring Harbor Laboratory Press, Cold Spring Harbor, N.Y.: Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning. Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s (x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques can be found in Kaufman and Rousseeuw; 1990, Finding Groups in Data: An Introduction to Cluster Analysis, Wiley, New York, N.Y.: Everitt, 1993, Cluster analysis (3d ed.), Wiley, New York, N. Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the model makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein: these gradient-boosting models are described in Boehmke, Bradley: Greenwell, Brandon (2019). “Gradient Boosting”. Hands-On Machine Learning with R. Chapman & Hall, pp. 221-245. ISBN 978-1-138-49568-5., which is hereby incorporated by reference in its entirety. In some embodiments, ensemble modeling techniques are used: these ensemble modeling techniques are described in the implementation of classification models herein and are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis is performed by a device executing one or more programs (e.g., one or more programs stored in the Non-Persistent Memory or in Persistent Memory) including instructions to perform the data analysis. In some embodiments, the data analysis is performed by a system comprising at least one processor (e.g., a processing core) and memory (e.g., one or more programs stored in Non-Persistent Memory or in the Persistent Memory) comprising instructions to perform the data analysis.

### Systems

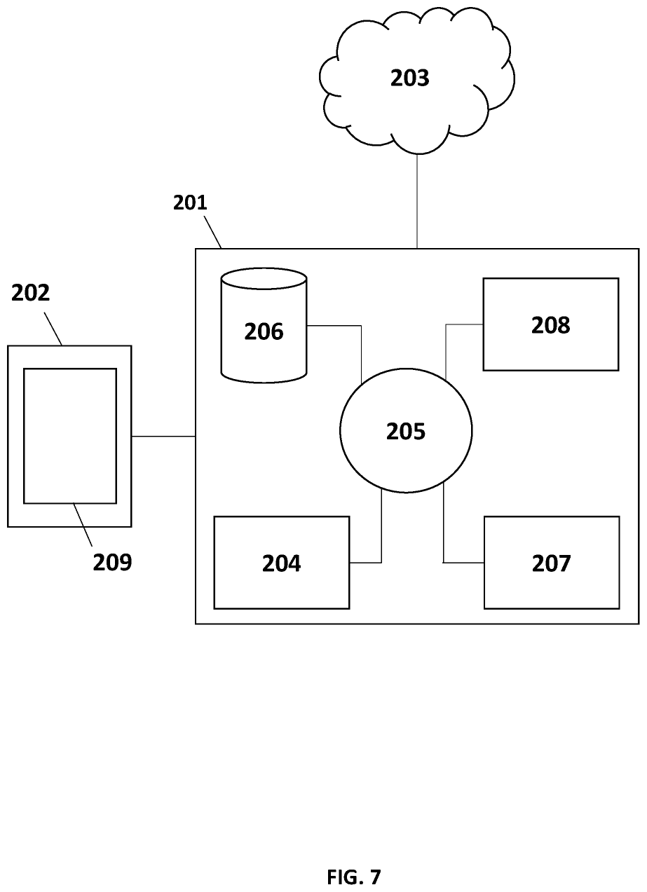

The present disclosure provides computer systems that are programmed to implement methods of the disclosure. FIG. 7 shows a computer system 201 that is programmed or otherwise configured to predict a disease (e.g., cancer or non-cancerous diseases), train a predictive model, generate a recommended therapeutic, generate and/or predict a longitudinal course of treatment of one or more subjects' disease, or any combination thereof methods, described elsewhere herein. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 204 (e.g., random-access memory, read-only memory, flash memory), electronic storage unit 206 (e.g., hard disk), communication interface 208 (e.g., network adapter) for communicating with one or more other systems, and peripheral devices 207, such as cache, other memory, data storage and/or electronic display adapters. The memory 204, storage unit 206, interface 208 and peripheral devices 207 are in communication with the CPU 205 through a communication bus (solid lines), such as a motherboard. The storage unit 206 can be a data storage unit (or data repository) for storing data. The computer system 201 can be operatively coupled to a computer network (“network”) 203 with the aid of the communication interface 208. The network 203 can be the Internet, an internet and/or extranet, or an intranet and/or extranet that is in communication with the Internet. The network 203 in some cases is a telecommunication and/or data network. The network 203 can include one or more computer servers, which can enable distributed computing, such as cloud computing. The network 203, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 204. The instructions can be directed to the CPU 205, which can subsequently program or otherwise configure the CPU 205 to implement methods of the present disclosure, described elsewhere herein. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 206 can store files, such as drivers, libraries, and saved programs. The storage unit 206 can store user data, e.g., disease predictions and/or one or more mammalian features and/or one or more non-mammalian features of the user and/or subjects' nucleic acid sequencing reads, user preferences, user programs, or any combination thereof. The computer system 201, in some cases can include one or more additional data storage units that are external to the computer system 201, such as located on a remote server that is in communication with the computer system 201 through an intranet or the Internet.

The computer system 201 can communicate with one or more remote computer systems through the network 203. For instance, the computer system 201 can communicate with a remote computer system of a user. Examples of remote computer systems may include personal computers (e.g., portable PC), slate or tablet PC's (e.g., Apple® iPad, Samsung® Galaxy Tab), telephones. Smart phones (e.g., Apple® iPhone, Android-enabled device, Blackberry®), or personal digital assistants. The user can access the computer system 201 via the network 203.

Methods as described herein can be implemented by way of machine (e.g., computer processor) executable code stored on an electronic storage location of the computer system 201, such as, for example, on the memory 204 or electronic storage unit 206. The machine executable or machine-readable code can be provided in the form of software. During use, the code can be executed by the processor 205. In some cases, the code can be retrieved from the storage unit 206 and stored on the memory 204 for ready access by the processor 205. In some situations, the electronic storage unit 206 can be precluded, and machine-executable instructions are stored on memory 204.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc, shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 202 that comprises a user interface (UI) 209 for providing, for example, a display for visualization of prediction results or an interface for training a predictive model, as described elsewhere herein. Examples of UI's include, without limitation, a graphical user interface (GUI) and web-based user interface.

Methods and systems of the present disclosure can be implemented by way of one or more algorithms and/or predictive models, described elsewhere herein. An algorithm and/or predictive model can be implemented by way of software upon execution by the central processing unit 205. The algorithm and/or predictive model may, for example, predict cancer of a subject or subjects, determine a tailored treatment and/or therapeutic to treat a subject's or subjects' disease (e.g., cancer as described elsewhere herein), predict a longitudinal course of a therapeutic to treat a subject's or one or more subjects' disease (e.g., cancer as described elsewhere herein), or any combination thereof.

### Embodiments

Numbered embodiment 1 comprises a method of determining a disease of a subject, comprising: providing a biological sample of a subject: enriching one or more nucleic acid molecules of the biological sample by affinity targeting of an epigenetic feature common to the one or more nucleic acid molecules; sequencing the enriched one or more nucleic acid molecules to generate one or more nucleic acid molecule sequencing reads; and determining the disease of the subject as an output of a predictive model when the predictive model is provided the enriched one or more nucleic acid molecules as an input. Numbered embodiment 2 comprises the method of numbered embodiment 1 wherein the one or more nucleic acid molecules comprise one or more mammalian nucleic acid molecules, one or more non-mammalian nucleic acid molecules, or a combination thereof. Numbered embodiment 3 comprises the method of numbered embodiment 1 or numbered embodiment 2 wherein the disease comprises cancer or a non-cancerous disease. Numbered embodiment 4 comprises the method of any one of numbered embodiment 1 to embodiment 3, wherein the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. Numbered embodiment 5 comprises the method of any one of numbered embodiment 1 to embodiment 4, wherein the non-cancerous disease comprises lupus erythematosus, type 2 diabetes, chronic obstructive pulmonary disease (COPD), sarcoidosis, or any combination thereof non-cancer diseases. Numbered embodiment 6 comprises the method of any one of numbered embodiment 1 to embodiment 5, further comprising filtering the one or more nucleic acid molecules sequencing reads to identify one or more non-mammalian sequencing reads and one or more mammalian sequencing reads. Numbered embodiment 7 comprises the method of any one of numbered embodiment 1 to embodiment 6, wherein the epigenetic feature comprises a nucleic acid epigenetic feature. Numbered embodiment 8 comprises the method of any one of numbered embodiment 1 to embodiment 7, wherein the epigenetic feature comprises a mammalian nucleic acid epigenetic feature or a non-mammalian nucleic acid epigenetic feature. Numbered embodiment 9 comprises the method of any one of numbered embodiment 1 to embodiment 8, wherein the non-mammalian nucleic acid epigenetic feature comprises phosphorothioate-linked nucleotides. Numbered embodiment 10 comprises the method of any one of numbered embodiment 1 to embodiment 9, wherein the biological sample comprises a tissue, liquid biopsy sample or a combination thereof. Numbered embodiment 11 comprises the method of any one of numbered embodiment 1 to embodiment 9, wherein the subject is human or a non-human mammal. Numbered embodiment 12 comprises the method of any one of numbered embodiment 1 to embodiment 11, wherein the one or more mammalian nucleic acid molecules, comprise DNA, RNA, cell-free DNA, cell-free RNA, exosomal DNA, exosomal RNA, or any combination thereof. Numbered embodiment 13 comprises the method of any one of numbered embodiment 1 to embodiment 12, wherein the one or more non-mammalian nucleic acid molecules, comprise DNA, RNA, cell-free DNA, cell-free RNA, exosomal DNA, exosomal RNA, or any combination thereof. Numbered embodiment 14 comprises the method of any one of numbered embodiment 1 to embodiment 13, wherein the affinity targeting of the nucleic acid epigenetic feature comprises concentrating the nucleic acid epigenetic feature. Numbered embodiment 15 comprises the method of any one of numbered embodiment 1 to embodiment 14, wherein the nucleic acid epigenetic feature comprises methylated CpG dinucleotides pairs, unmethylated CpG dinucleotide pairs, or a combination thereof. Numbered embodiment 16 comprises the method of any one of numbered embodiment 1 to embodiment 15, wherein the nucleic acid epigenetic feature comprises nucleobases 5-methylcytosine. 5-hydroxymethylcytosine. N4-acetylcytosine, N6-methyladenine, or any combination thereof. Numbered embodiment 17 comprises the method of any one of numbered embodiment 1 to embodiment 16, wherein the affinity targeting utilizes specific affinity reagents to bind to the epigenetic feature. Numbered embodiment 18 comprises the method of any one of numbered embodiment 1 to embodiment 17, wherein the specific affinity reagents comprise streptavidin. NeutrAvidin, polyclonal, monoclonal, recombinant antibodies, aptamers, recombinant epigenetic proteins, or any combination thereof. Numbered embodiment 19 comprises the method of any one of numbered embodiment 1 to embodiment 18, wherein the recombinant epigenetic proteins comprise epigenetic readers, writers, erasers, or any combination thereof. Numbered embodiment 20 comprises the method of any one of numbered embodiment 1 to embodiment 19, wherein the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom. Numbered embodiment 21 comprises the method of any one of numbered embodiment 1 to embodiment 20, wherein the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. Numbered embodiment 22 comprises the method of any one of numbered embodiment 1 to embodiment 21, wherein the epigenetic readers comprise the microbial proteins Dam, CcrM, ModA13, SpnD39III, Dcm, JHP1050, M2.Hpy.AII, or the recombinant methyl-binding domains derived therefrom. Numbered embodiment 23 comprises the method of any one of numbered embodiment 1 to embodiment 22, wherein the epigenetic writers and erasers are catalytically inactive. Numbered embodiment 24 comprises the method of any one of numbered embodiment 1 to embodiment 23, wherein the epigenetic readers, writers, and erasers comprise an epitope tag. Numbered embodiment 25 comprises the method of any one of numbered embodiment 1 to embodiment 24, wherein the epitope tag comprises a N- or C-terminal 6x-histidine tag, green fluorescent protein (MA), myc, hemagglutinin (HA), Fc fusion, molecular recognition motif, or any combination thereof. Numbered embodiment 26 comprises the method of any one of numbered embodiment 1 to embodiment 25, wherein the molecular recognition motif comprises a birA or sortase motif. Numbered embodiment 27 comprises the method of any one of numbered embodiment 1 to embodiment 26, further comprising concentrating the one or more mammalian and non-mammalian nucleic acid molecules by a solid support, wherein the solid support comprises immobilized complementary antibodies to the epitope tag. Numbered embodiment 28 comprises the method of any one of numbered embodiment 1 to embodiment 27, wherein the specific affinity reagents comprise a region to recognize and bind to the epigenetic feature. Numbered embodiment 29 comprises the method of any one of numbered embodiment 1 to embodiment 28, wherein affinity targeting comprises incubating the biological sample with a solid support comprising a plurality of immobilized affinity agents. Numbered embodiment 30 comprises the method of any one of numbered embodiment 1 to embodiment 29, wherein the plurality of immobilized affinity agents comprises a region that will bind to the epigenetic feature. Numbered embodiment 31 comprises the method of any one of numbered embodiment 1 to embodiment 30, wherein the solid support comprises a magnetic bead, an agarose bead, non-magnetic latex, functionalized Sepharose, pH-sensitive polymers, or any combination thereof. Numbered embodiment 32 comprises the method of any one of numbered embodiment 1 to embodiment 31, wherein the filtering comprises filtering the one or more mammalian sequencing reads and the one or more non-mammalian sequencing reads against a genome database. Numbered embodiment 33 comprises the method of any one of numbered embodiment 1 to embodiment 32, wherein the genome database is a human genome database. Numbered embodiment 34 comprises the method of any one of numbered embodiment 1 to embodiment 33, wherein the predictive model is trained with one or more mammalian, one or more non-mammalian, or a combination thereof features determined from one or more subjects' one or more nucleic acid molecules of a biological sample and a corresponding disease of the one or more subjects. Numbered embodiment 35 comprises the method of any one of numbered embodiment 1 to embodiment 34, wherein the one or more mammalian feature comprises mammalian genomic coordinates or annotated genomic loci and a number of sequencing reads associated therewith. Numbered embodiment 36 comprises the method of any one of numbered embodiment 1 to embodiment 35, wherein the one or more mammalian feature comprises mammalian functional gene and biochemical pathway abundances. Numbered embodiment 37 comprises the method of any one of numbered embodiment 1 to embodiment 36, wherein the one or more non-mammalian feature comprises microbial taxonomic assignments and a number of sequencing reads associated therewith. Numbered embodiment 38 comprises the method of any one of numbered embodiment 1 to embodiment 37, wherein the one or more non-mammalian features comprises microbial functional gene and biochemical pathway abundances. Numbered embodiment 39 comprises the method of any one of numbered embodiment 1 to embodiment 38, wherein the liquid biopsy sample comprises plasma, serum, whole blood, urine, cerebral spinal fluid, saliva sweat, tears, exhaled breath condensate, or any combination thereof. Numbered embodiment 40 comprises the method of any one of numbered embodiment 1 to embodiment 39, wherein the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when one or more nucleic acid molecules of the biological sample are not enriched. Numbered embodiment 41 comprises the method of any one of numbered embodiment 1 to embodiment 40, wherein the predictive model comprises an area under the curve of at least about 0.70, at least about 0.80, at least about 0.85, at least about 0.90, or at least about 0.95 when determining the disease of the subject. Numbered embodiment 42 comprises the method of any one of numbered embodiment 1 to embodiment 41, wherein an output of the trained predictive model comprises an analysis of a combination of the one or more mammalian features and the one or more non-mammalian features abundance. Numbered embodiment 43 comprises the method of any one of numbered embodiment 1 to embodiment 42, wherein an input of the trained predictive model comprises epigenomic abundance information from one or more of the following kingdoms of life: mammalian, bacterial, archaeal, fungal, and/or viral. Numbered embodiment 44 comprises the method of any one of numbered embodiment 1 to embodiment 43, wherein the predictive model is further trained with a tissue-specific location of the disease. Numbered embodiment 45 comprises the method of any one of numbered embodiment 1 to embodiment 44, wherein the predictive model is further trained with the cancer's type, subtype, stage, prognosis, or any combination thereof. Numbered embodiment 46 comprises the method of any one of numbered embodiment 1 to embodiment 45, wherein the predictive model outputs the cancer's type, subtype, stage, prognosis, or any combination thereof when provided the subject's nucleic acid sequencing reads of the biological sample. Numbered embodiment 47 comprises the method of any one of numbered embodiment 1 to embodiment 46, wherein the predictive model outputs the subject's cancer therapy response. Numbered embodiment 48 comprises the method of any one of numbered embodiment 1 to embodiment 47, wherein the trained predictive model outputs a therapy for the subject that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the subject. Numbered embodiment 49 comprises the method of any one of numbered embodiment 1 to embodiment 48, wherein the trained predictive model outputs a longitudinal model of the subject's cancer in response to a therapy, an adjustment to a therapy to treat the subject's cancer, or a combination thereof. Numbered embodiment 50 comprises the method of any one of numbered embodiment 1 to embodiment 49, wherein the predictive model removes contaminate non-mammalian features while selectively retaining other non-contaminate non-mammalian features. Numbered embodiment 51 comprises the method of any one of numbered embodiment 1 to embodiment 50, wherein enriching reduces a total of the one or more nucleic acid molecules by at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99%.

Numbered embodiment 51 comprises a method of training a predictive model, comprising: providing a biological sample of one or more subjects with a disease; enriching the biological sample of the one or more subjects by affinity targeting of an epigenetic feature common to one or more nucleic acid molecules of the biological sample; sequencing the enriched one or more nucleic acid molecules to generate one or more nucleic acid molecule sequencing reads; and training the predictive model with one or more features of the one or more nucleic acid molecule sequencing reads and the disease of the one or more subjects. Numbered embodiment 52 comprises the method of embodiment 51, wherein the epigenetic feature comprises a mammalian epigenetic feature or a non-mammalian epigenetic feature. Numbered embodiment 53 comprises the method of embodiment 51 or embodiment 52, wherein the one or more features comprise one or more disease features. Numbered embodiment 54 comprises the method of any one of numbered embodiment 51 to embodiment 53, wherein the trained predictive model determines a disease of another one or more subjects that differ from the one or more subjects when the trained predictive model is provided the another one or more subjects' nucleic acid sequencing reads of a biological sample. Numbered embodiment 55 comprises the method of any one of numbered embodiment 51 to embodiment 54, wherein the one or more nucleic acid molecules comprise one or more mammalian nucleic acid molecules, one or more non-mammalian nucleic acid molecules, or a combination thereof. Numbered embodiment 56 comprises the method of any one of numbered embodiment 51 to embodiment 55, further comprising filtering the one or more nucleic acid sequencing reads to identify one or more non-mammalian sequencing reads, the one or more mammalian sequencing reads, or a combination thereof. Numbered embodiment 57 comprises the method of any one of numbered embodiment 51 to embodiment 56, wherein the epigenetic feature comprises a nucleic acid epigenetic feature. Numbered embodiment 58 comprises the method of any one of numbered embodiment 51 to embodiment 57, wherein the biological sample comprises a tissue, liquid biopsy sample or a combination thereof. Numbered embodiment 58 comprises the method of any one of numbered embodiment 51 to embodiment 57, wherein the one or more subjects are human or a non-human mammal. Numbered embodiment 59 comprises the method of any one of numbered embodiment 51 to embodiment 58, wherein the one or more mammalian nucleic acid molecules comprise DNA, RNA, cell-free DNA, cell-free RNA, exosomal DNA, exosomal RNA, or any combination thereof. Numbered embodiment 60 comprises the method of any one of numbered embodiment 51 to embodiment 59, wherein the one or more non-mammalian nucleic acid molecules comprise DNA, RNA, cell-free DNA, cell-free RNA, exosomal DNA, exosomal RNA, or any combination thereof. Numbered embodiment 61 comprises the method of any one of numbered embodiment 51 to embodiment 60, wherein the affinity targeting of the nucleic acid epigenetic feature comprises concentrating the nucleic acid epigenetic feature. Numbered embodiment 62 comprises the method of any one of numbered embodiment 51 to embodiment 61, wherein the nucleic acid epigenetic feature comprises methylated CpG dinucleotides pairs, unmethylated CpG dinucleotide pairs, or a combination thereof. Numbered embodiment 63 comprises the method of any one of numbered embodiment 51 to embodiment 62, wherein the nucleic acid epigenetic feature comprises nucleobases 5-methylcytosine, 5-hydroxymethylcytosine, N4-acetylcytosine, N6-methyladenine, or any combination thereof. Numbered embodiment 64 comprises the method of any one of numbered embodiment 51 to embodiment 63, wherein the affinity targeting utilizes specific affinity reagents to bind to the epigenetic feature. Numbered embodiment 65 comprises the method of any one of numbered embodiment 51 to embodiment 64, wherein the specific affinity reagents comprise streptavidin, NeutrAvidin, polyclonal, monoclonal, recombinant antibodies, aptamers, recombinant epigenetic proteins, or any combination thereof. Numbered embodiment 66 comprises the method of any one of numbered embodiment 51 to embodiment 65, wherein the recombinant epigenetic proteins comprise epigenetic readers, writers, erasers, or any combination thereof. Numbered embodiment 67 comprises the method of any one of numbered embodiment 51 to embodiment 66, wherein the epigenetic readers comprise recombinant methyl-CpG binding proteins Mecp2, Mbd1-6, SETDB1, SETDB2, TIP5/BAZ2A. Zbtb38, Kaiso, Zbtb4, Np95, Np97 or the recombinant methyl-binding domains derived therefrom. Numbered embodiment 68 comprises the method of any one of numbered embodiment 51 to embodiment 67, wherein the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. Numbered embodiment 69 comprises the method of any one of numbered embodiment 51 to embodiment 68, wherein the epigenetic writers and erasers are catalytically inactive. Numbered embodiment 70 comprises the method of any one of numbered embodiment 51 to embodiment 69, wherein the epigenetic readers, writers, and erasers comprise an epitope tag. Numbered embodiment 71 comprises the method of any one of numbered embodiment 51 to embodiment 70, wherein the epitope tag comprises a N- or C-terminal 6x-histidine tag, green fluorescent protein (MA), myc, hemagglutinin (HA), Fc fusion, molecular recognition motif, or any combination thereof. Numbered embodiment 72 comprises the method of any one of numbered embodiment 51 to embodiment 71, wherein the molecular recognition motif comprises a birA or sortase motif. Numbered embodiment 73 comprises the method of any one of numbered embodiment 51 to embodiment 72, further comprising concentrating the one or more mammalian and the one or more non-mammalian nucleic acid molecules by a solid support, wherein the solid support comprises immobilized complementary antibodies to the epitope tag. Numbered embodiment 74 comprises the method of any one of numbered embodiment 51 to embodiment 73, wherein the specific affinity reagents comprise a region to recognize and bind to the epigenetic feature. Numbered embodiment 75 comprises the method of any one of numbered embodiment 51 to embodiment 74, wherein affinity targeting comprises incubating the biological sample with a solid support comprising a plurality of immobilized affinity agents. Numbered embodiment 76 comprises the method of any one of numbered embodiment 51 to embodiment 75, wherein the plurality of immobilized affinity agents comprises a region that will bind to the epigenetic feature. Numbered embodiment 77 comprises the method of any one of numbered embodiment 51 to embodiment 76, wherein the solid support comprises a magnetic bead, an agarose bead, non-magnetic latex, functionalized Sepharose, pH-sensitive polymers, or any combination thereof. Numbered embodiment 78 comprises the method of any one of numbered embodiment 51 to embodiment 77, wherein filtering comprises filtering the one or more mammalian and non-mammalian sequencing reads against a genome database. Numbered embodiment 79 comprises the method of any one of numbered embodiment 51 to embodiment 78, wherein the genome database is a human genome database. Numbered embodiment 80 comprises the method of any one of numbered embodiment 51 to embodiment 79, wherein the one or more features comprise one or more mammalian features, one or more non-mammalian features, or a combination thereof features. Numbered embodiment 81 comprises the method of any one of numbered embodiment 51 to embodiment 80, wherein the one or more mammalian features comprise mammalian genomic coordinates or annotated genomic loci and a number of sequencing reads associated therewith. Numbered embodiment 82 comprises the method of any one of numbered embodiment 51 to embodiment 81, wherein the one or more mammalian features comprise mammalian functional gene and biochemical pathway abundances. Numbered embodiment 83 comprises the method of any one of numbered embodiment 51 to embodiment 82, wherein the one or more non-mammalian features comprise microbial taxonomic assignments and a number of sequencing reads associated therewith. Numbered embodiment 84 comprises the method of any one of numbered embodiment 51 to embodiment 83, wherein the one or more non-mammalian features comprises microbial functional gene and biochemical pathway abundances. Numbered embodiment 85 comprises the method of any one of numbered embodiment 51 to embodiment 84, wherein the liquid biopsy sample comprises plasma, serum, whole blood, urine, cerebral spinal fluid, saliva, sweat, tears, exhaled breath condensate, or any combination thereof. Numbered embodiment 86 comprises the method of any one of numbered embodiment 51 to embodiment 85, wherein the disease comprises cancer or non-cancerous disease. Numbered embodiment 87 comprises the method of any one of numbered embodiment 51 to embodiment 86, wherein the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when one or more nucleic acid molecules of the biological sample are not enriched. Numbered embodiment 88 comprises the method of any one of numbered embodiment 51 to embodiment 87, wherein the predictive model comprises an area under the curve of at least about 0.70, at least about 0.80, at least about 0.85, at least about 0.90, or at least about 0.95 when determining the disease of the another one or more subjects. Numbered embodiment 89 comprises the method of any one of numbered embodiment 51 to embodiment 88, wherein the non-mammalian nucleic acid epigenetic feature comprises phosphorothioate-linked nucleotides. Numbered embodiment 90 comprises the method of any one of numbered embodiment 51 to embodiment 89, wherein the epigenetic readers comprise the microbial proteins Dam, CcrM, ModA13, SpnD39III, Dcm, JHP1050, M2.Hpy.AII, or the recombinant methyl-binding domains derived therefrom. Numbered embodiment 91 comprises the method of any one of numbered embodiment 51 to embodiment 90, wherein an output of the trained predictive model comprises an analysis of a combination of the one or more mammalian features and the one or more non-mammalian features abundance. Numbered embodiment 92 comprises the method of any one of numbered embodiment 51 to embodiment 91, wherein an input of the trained predictive model comprises epigenomic abundance information from one or more of the following kingdoms of life: mammalian, bacterial, archaeal, fungal, and/or viral. Numbered embodiment 93 comprises the method of any one of numbered embodiment 51 to embodiment 92, wherein the predictive model is further trained with a tissue-specific location of the disease. Numbered embodiment 94 comprises the method of any one of numbered embodiment 51 to embodiment 93, wherein the predictive model is further trained with the cancer's type, subtype, stage, prognosis, or any combination thereof. Numbered embodiment 95 comprises the method of any one of numbered embodiment 51 to embodiment 94, wherein the predictive model outputs the cancer's type, subtype, stage, prognosis, or any combination thereof when provided the another one or more subjects' nucleic acid sequencing reads of the biological sample. Numbered embodiment 96 comprises the method of any one of numbered embodiment 51 to embodiment 95, wherein the trained predictive model outputs the another one or more subjects' cancer therapy response. Numbered embodiment 97 comprises the method of any one of numbered embodiment 51 to embodiment 96, wherein the trained predictive model outputs a therapy for the another one or more subjects that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the another one or more subjects. Numbered embodiment 98 comprises the method of any one of numbered embodiment 51 to embodiment 97, wherein the trained predictive model outputs a longitudinal model of the another one or more subjects' cancers in response to a therapy, an adjustment to a therapy to treat the subject's cancer, or a combination thereof. Numbered embodiment 99 comprises the method of any one of numbered embodiment 51 to embodiment 98, wherein the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma testicular germ cell tumors, thymoma, thyroid carcinoma, uterine carcinosarcoma, uterine corpus endometrial carcinoma, uveal melanoma, or any combination thereof. Numbered embodiment 100 comprises the method of any one of numbered embodiment 51 to embodiment 99, wherein the predictive model is configured to remove contaminate non-mammalian features while selectively retaining other non-contaminate non-mammalian features. Numbered embodiment 101 comprises the method of any one of numbered embodiment 51 to embodiment 100, wherein the non-cancerous disease comprises lupus erythematosus, type 2 diabetes, chronic obstructive pulmonary disease (COPD), sarcoidosis, or any combination thereof non-cancer diseases. Numbered embodiment 102 comprises the method of any one of numbered embodiment 51 to embodiment 101, wherein the enriching reduces a total of the one or more nucleic acid molecules by at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99%.

Numbered embodiment 103 comprises a computer system to determine a disease of a subject, comprising: one or more processors; and a non-transient computer readable storage medium including software, wherein the software comprises executable instruction that, as a result of execution, cause the one or more processors of the computer system to: (i) receive a subject's one or more nucleic acid molecule sequencing reads of one or more nucleic acid molecules of a biological sample, wherein the one or more nucleic acid molecules are enriched by affinity targeting of an epigenetic feature common to the one or more nucleic acid molecules; and (ii) determine a disease of the subject as an output of a predictive model when the predictive model is provided the one or more nucleic acid molecule sequencing. Numbered embodiment 104 comprises the system of embodiment 103, wherein the one or more nucleic acid molecules comprise one or more mammalian nucleic acid molecules, one or more non-mammalian nucleic acid molecules, or a combination thereof. Numbered embodiment 105 comprises the system of embodiment 103 or embodiment 104, wherein the disease comprises cancer or a non-cancerous disease. Numbered embodiment 106 comprises the system of any one of numbered embodiment 103 to embodiment 105, wherein the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. Numbered embodiment 107 comprises the system of any one of numbered embodiment 103 to embodiment 106, wherein the non-cancerous disease comprises lupus erythematosus, type 2 diabetes, chronic obstructive pulmonary disease (COPD), sarcoidosis, or any combination thereof non-cancer diseases. Numbered embodiment 108 comprises the system of any one of numbered embodiment 103 to embodiment 107, wherein the executable instruction further comprise filter the one or more nucleic acid molecules sequencing reads to identify one or more non-mammalian sequencing reads and one or more mammalian sequencing reads. Numbered embodiment 109 comprises the system of any one of numbered embodiment 103 to embodiment 108, wherein the epigenetic feature comprises a nucleic acid epigenetic feature. Numbered embodiment 110 comprises the system of any one of numbered embodiment 103 to embodiment 109, wherein the epigenetic feature comprises a mammalian nucleic acid epigenetic feature or a non-mammalian nucleic acid epigenetic feature. Numbered embodiment 111 comprises the system of any one of numbered embodiment 103 to embodiment 110, wherein the non-mammalian nucleic acid epigenetic feature comprises phosphorothioate-linked nucleotides. Numbered embodiment 112 comprises the system of any one of numbered embodiment 103 to embodiment 111, wherein the biological sample comprises a tissue, liquid biopsy sample or a combination thereof. Numbered embodiment 113 comprises the system of any one of numbered embodiment 103 to embodiment 112, wherein the subject is human or a non-human mammal. Numbered embodiment 114 comprises the system of any one of numbered embodiment 103 to embodiment 113, wherein the one or more mammalian nucleic acid molecules, comprise DNA, RNA, cell-free DNA, cell-free RNA, exosomal DNA, exosomal RNA, or any combination thereof. Numbered embodiment 115 comprises the system of any one of numbered embodiment 103 to embodiment 114, wherein the one or more non-mammalian nucleic acid molecules, comprise DNA, RNA, cell-free DNA, cell-free RNA, exosomal DNA, exosomal RNA, or any combination thereof. Numbered embodiment 116 comprises the system of any one of numbered embodiment 103 to embodiment 115, wherein the affinity targeting of the nucleic acid epigenetic feature comprises concentrating the nucleic acid epigenetic feature. Numbered embodiment 117 comprises the system of any one of numbered embodiment 103 to embodiment 116, wherein the nucleic acid epigenetic feature comprises methylated CpG dinucleotides pairs, unmethylated CpG dinucleotide pairs, or a combination thereof. Numbered embodiment 118 comprises the system of any one of numbered embodiment 103 to embodiment 117, wherein the nucleic acid epigenetic feature comprises nucleobases 5-methylcytosine, 5-hydroxymethylcytosine, N4-acetylcytosine, N6-methyladenine, or any combination thereof. Numbered embodiment 119 comprises the system of any one of numbered embodiment 103 to embodiment 118, wherein affinity targeting utilizes specific affinity reagents to bind to the epigenetic feature. Numbered embodiment 120 comprises the system of any one of numbered embodiment 103 to embodiment 119, wherein the specific affinity reagents comprise streptavidin, NeutrAvidin, polyclonal, monoclonal, recombinant antibodies, aptamers, recombinant epigenetic proteins, or any combination thereof. Numbered embodiment 121 comprises the system of any one of numbered embodiment 103 to embodiment 120, wherein the recombinant epigenetic proteins comprise epigenetic readers, writers, erasers, or any combination thereof. Numbered embodiment 122 comprises the system of any one of numbered embodiment 103 to embodiment 121, wherein the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom. Numbered embodiment 123 comprises the system of any one of numbered embodiment 103 to embodiment 122, wherein the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom. Numbered embodiment 124 comprises the system of any one of numbered embodiment 103 to embodiment 123, wherein the epigenetic readers comprise the microbial proteins Dam, CcrM, ModA13, SpnD39III, Dem, JHP1050, M2.Hpy.AII, or the recombinant methyl-binding domains derived therefrom. Numbered embodiment 125 comprises the system of any one of numbered embodiment 103 to embodiment 124, wherein the epigenetic writers and erasers are catalytically inactive. Numbered embodiment 126 comprises the system of any one of numbered embodiment 103 to embodiment 125, wherein the epigenetic readers, writers, and erasers comprise an epitope tag. Numbered embodiment 127 comprises the system of any one of numbered embodiment 103 to embodiment 126, wherein the epitope tag comprises a N- or C-terminal 6x-histidine tag, green fluorescent protein (MA), myc, hemagglutinin (HA), Fc fusion, molecular recognition motif, or any combination thereof. Numbered embodiment 128 comprises the system of any one of numbered embodiment 103 to embodiment 127, wherein the molecular recognition motif comprises a birA or sortase motif. Numbered embodiment 129 comprises the system of any one of numbered embodiment 103 to embodiment 128, further comprising concentrating the one or more mammalian and non-mammalian nucleic acid molecules by a solid support, wherein the solid support comprises immobilized complementary antibodies to the epitope tag. Numbered embodiment 130 comprises the system of any one of numbered embodiment 103 to embodiment 129, wherein the specific affinity reagents comprise a region to recognize and bind to the epigenetic feature. Numbered embodiment 131 comprises the system of any one of numbered embodiment 103 to embodiment 130, wherein affinity targeting comprises incubating the biological sample with a solid support comprising a plurality of immobilized affinity agents. Numbered embodiment 132 comprises the system of any one of numbered embodiment 103 to embodiment 131, wherein the plurality of immobilized affinity agents comprises a region that will bind to the epigenetic feature. Numbered embodiment 133 comprises the system of any one of numbered embodiment 103 to embodiment 132, wherein the solid support comprises a magnetic bead, an agarose bead, non-magnetic latex, functionalized Sepharose, pH-sensitive polymers, or any combination thereof. Numbered embodiment 134 comprises the system of any one of numbered embodiment 103 to embodiment 133, wherein filtering comprises filtering the one or more mammalian sequencing reads and the one or more non-mammalian sequencing reads against a genome database. Numbered embodiment 135 comprises the system of any one of numbered embodiment 103 to embodiment 134, wherein the genome database is a human genome database. Numbered embodiment 136 comprises the system of any one of numbered embodiment 103 to embodiment 135, wherein the predictive model is trained with one or more mammalian, one or more non-mammalian, or a combination thereof features determined from one or more subjects' one or more nucleic acid molecules of a biological sample and a corresponding disease of the one or more subjects. Numbered embodiment 137 comprises the system of any one of numbered embodiment 103 to embodiment 136, wherein the one or more mammalian feature comprises mammalian genomic coordinates or annotated genomic loci and a number of sequencing reads associated therewith. Numbered embodiment 138 comprises the system of any one of numbered embodiment 103 to embodiment 137, wherein the one or more mammalian feature comprises mammalian functional gene and biochemical pathway abundances. Numbered embodiment 139 comprises the system of any one of numbered embodiment 103 to embodiment 138, wherein the one or more non-mammalian feature comprise microbial taxonomic assignments and a number of sequencing reads associated therewith. Numbered embodiment 140 comprises the system of any one of numbered embodiment 103 to embodiment 139, wherein the one or more non-mammalian features comprise microbial functional gene and biochemical pathway abundances Numbered embodiment 141 comprises the system of any one of numbered embodiment 103 to embodiment 140, wherein the liquid biopsy sample comprises plasma, serum, whole blood, urine, cerebral spinal fluid, saliva, sweat, tears, exhaled breath condensate, or any combination thereof Numbered embodiment 142 comprises the system of any one of numbered embodiment 103 to embodiment 141, wherein the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when one or more nucleic acid molecules of the biological sample are not enriched. Numbered embodiment 143 comprises the system of any one of numbered embodiment 103 to embodiment 142, wherein the predictive model comprises an area under the curve of at least about 0.70, at least about 0.80, at least about 0.85, at least about 0.90, or at least about 0.95 when determining the disease of the subject. Numbered embodiment 144 comprises the system of any one of numbered embodiment 103 to embodiment 143, wherein an output of the trained predictive model comprises an analysis of a combination of the one or more mammalian features and the one or more non-mammalian features abundance. Numbered embodiment 145 comprises the system of any one of numbered embodiment 103 to embodiment 144, wherein an input of the trained predictive model comprises epigenomic abundance information from one or more of the following kingdoms of life: mammalian, bacterial, archaeal, fungal, and/or viral. Numbered embodiment 146 comprises the system of any one of numbered embodiment 103 to embodiment 145, wherein the predictive model is further trained with a tissue-specific location of the disease. Numbered embodiment 147 comprises the system of any one of numbered embodiment 103 to embodiment 146, wherein the predictive model is further trained with the cancer's type, subtype, stage, prognosis, or any combination thereof. Numbered embodiment 148 comprises the system of any one of numbered embodiment 103 to embodiment 147, wherein the predictive model outputs the cancer's type, subtype, stage, prognosis, or any combination thereof when provided the subject's nucleic acid sequencing reads of the biological sample. Numbered embodiment 149 comprises the system of any one of numbered embodiment 103 to embodiment 148, wherein the predictive model outputs the subject's cancer therapy response. Numbered embodiment 150 comprises the system of any one of numbered embodiment 103 to embodiment 149, wherein the trained predictive model outputs a therapy for the subject that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the subject. Numbered embodiment 151 comprises the system of any one of numbered embodiment 103 to embodiment 150, wherein the trained predictive model outputs a longitudinal model of the subject's cancer in response to a therapy, an adjustment to a therapy to treat the subject's cancer, or a combination thereof. Numbered embodiment 152 comprises the system of any one of numbered embodiment 103 to embodiment 151, wherein the predictive model removes contaminate non-mammalian features while selectively retaining other non-contaminate non-mammalian features. Numbered embodiment 153 comprises the system of any one of numbered embodiment 103 to embodiment 152, wherein the enriched nucleic acids comprise a reduction of at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99% of the one or more nucleic acid molecules prior to enrichment.

### EXAMPLES

### Example 1:5-hydroxymethylcytosine microbial epigenetic biomarker discovery and cancer diagnostic model evaluation

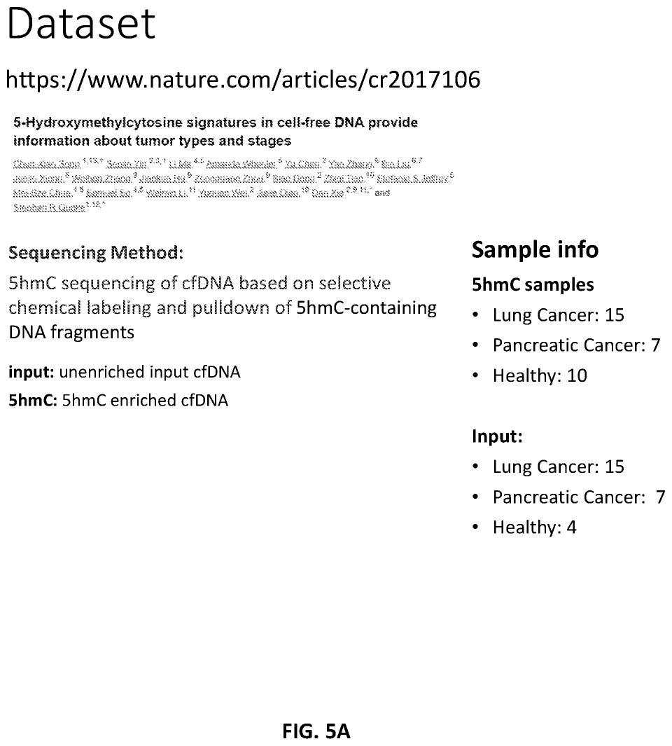

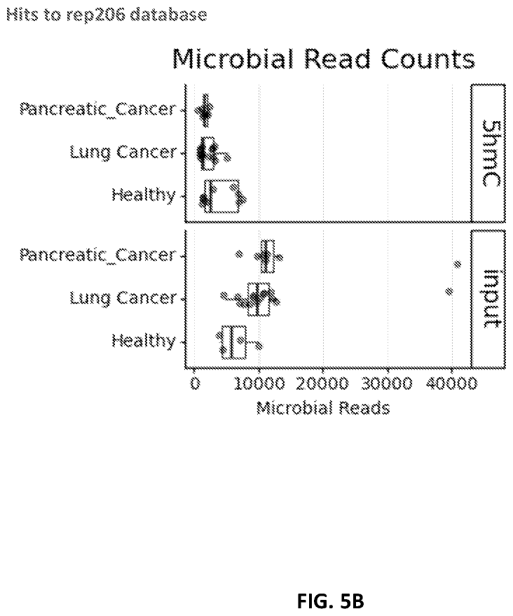

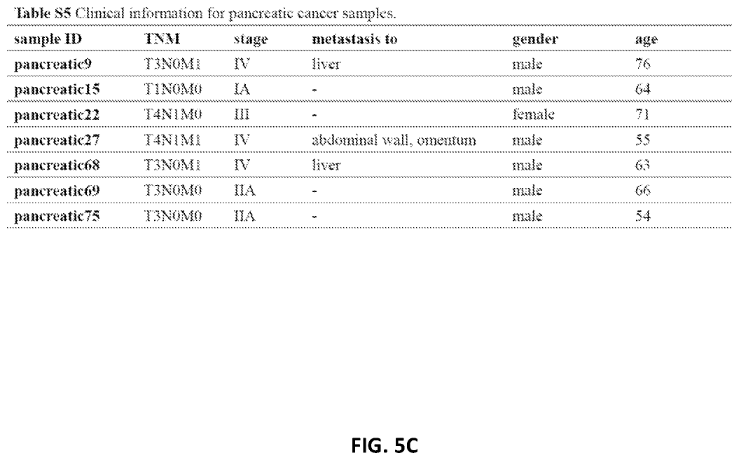

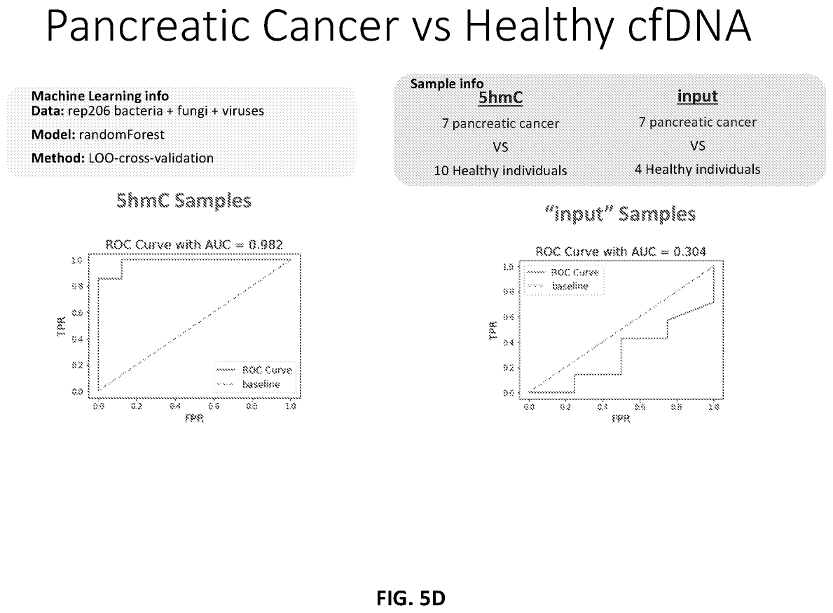

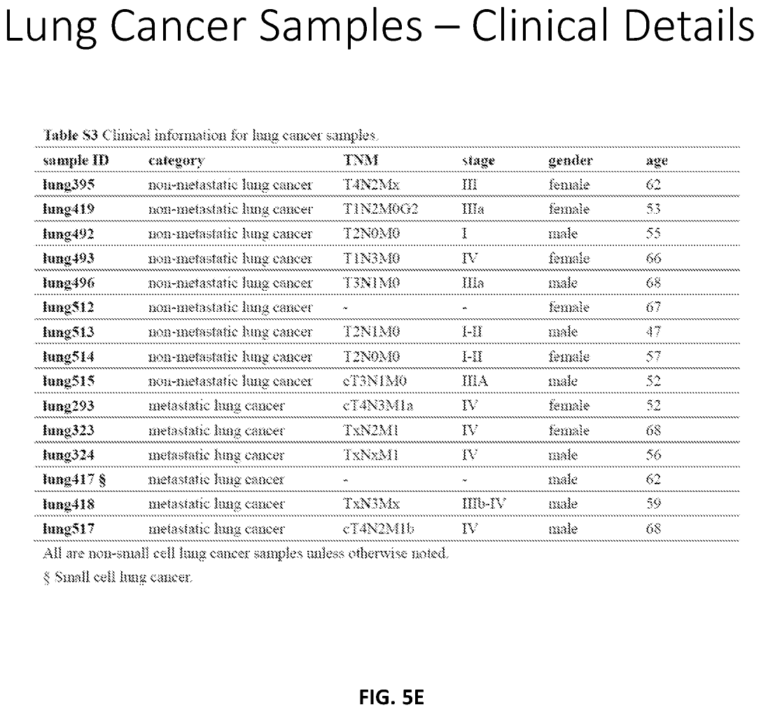

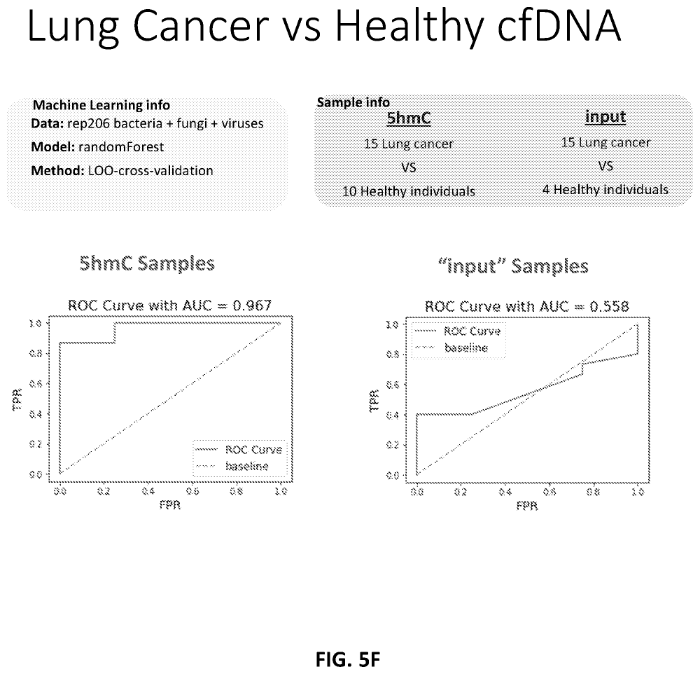

FIGS. 5A-5D show experimental parameters and resulting classification accuracy of a study of 5-hydroxymethylcytosine (5hmC) microbial epigenetic biomarker discovery and cancer diagnostic model evaluation. FIG. 5A shows the cell-free DNA study whence the 5-hydroxylmethylcytosine-enrichment sequencing data was obtained, and sample types present in the sequencing data. The non-human sequencing data obtained was then aligned to a reference database of microbial genomes (“rep206”). The dataset of the alignment of the non-human reads is shown in FIG. 5B. FIG. 5C shows the clinical details of the pancreatic cancer samples present in the aligned dataset. A machine learning model was then trained on 5hmC-enriched microbial nucleic acids from pancreatic cancer patients and healthy individuals (“5hmC Samples” ROC Curve, left: the “input Samples” ROC Curve was generated from the unenriched nucleic acids) shown in FIG. 5D. Due to the small sample number, leave-one-out (LOO)-cross validation was performed in lieu of a more traditional 70/30 train-test split of sample feature sets. FIG. 5E shows the clinical details of the lung cancer samples present in the dataset. FIG. 5F shows the performance of a machine learning model trained on 5hmC-enriched microbial nucleic acids from lunger cancer patients and healthy individuals. As in FIG. 5D. LOO was utilized to develop the lung cancer classifier.

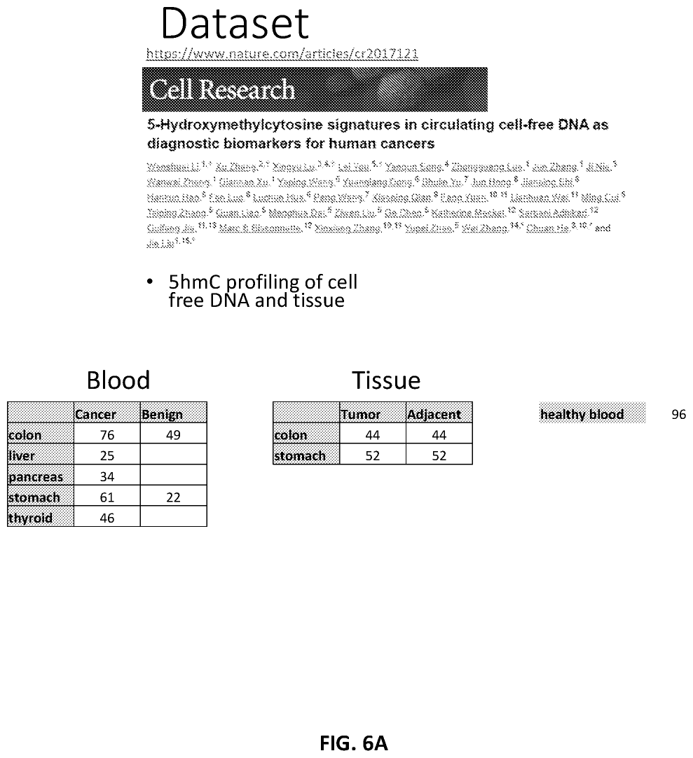

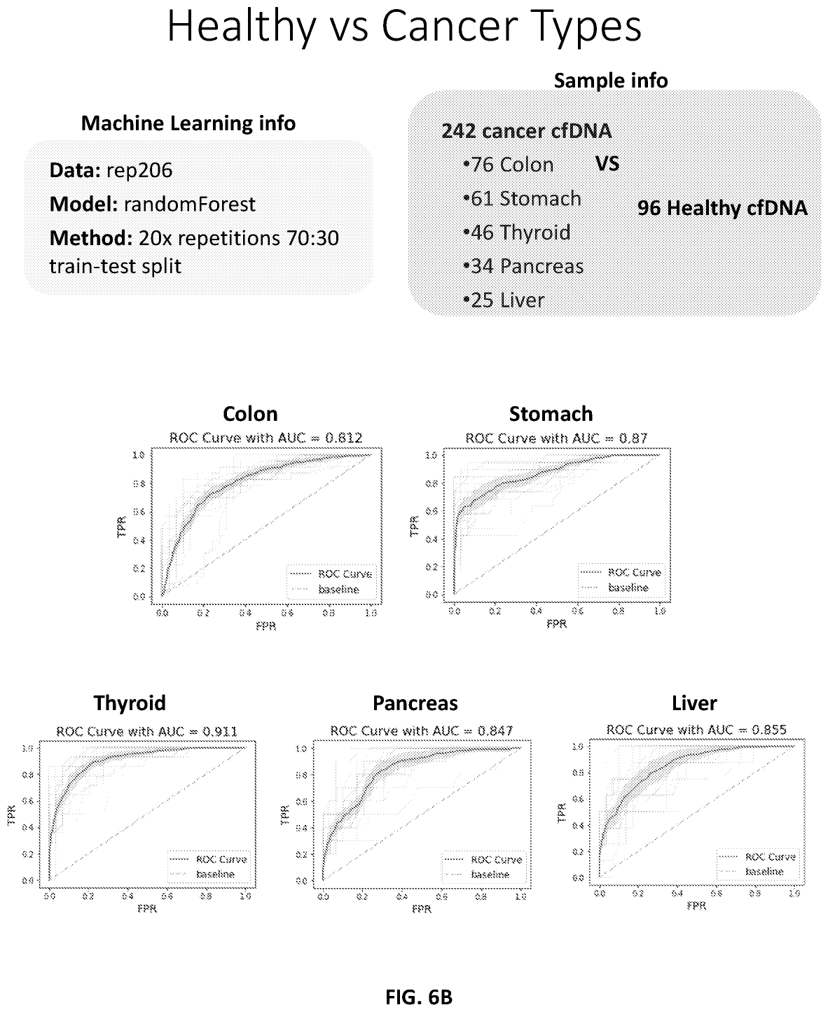

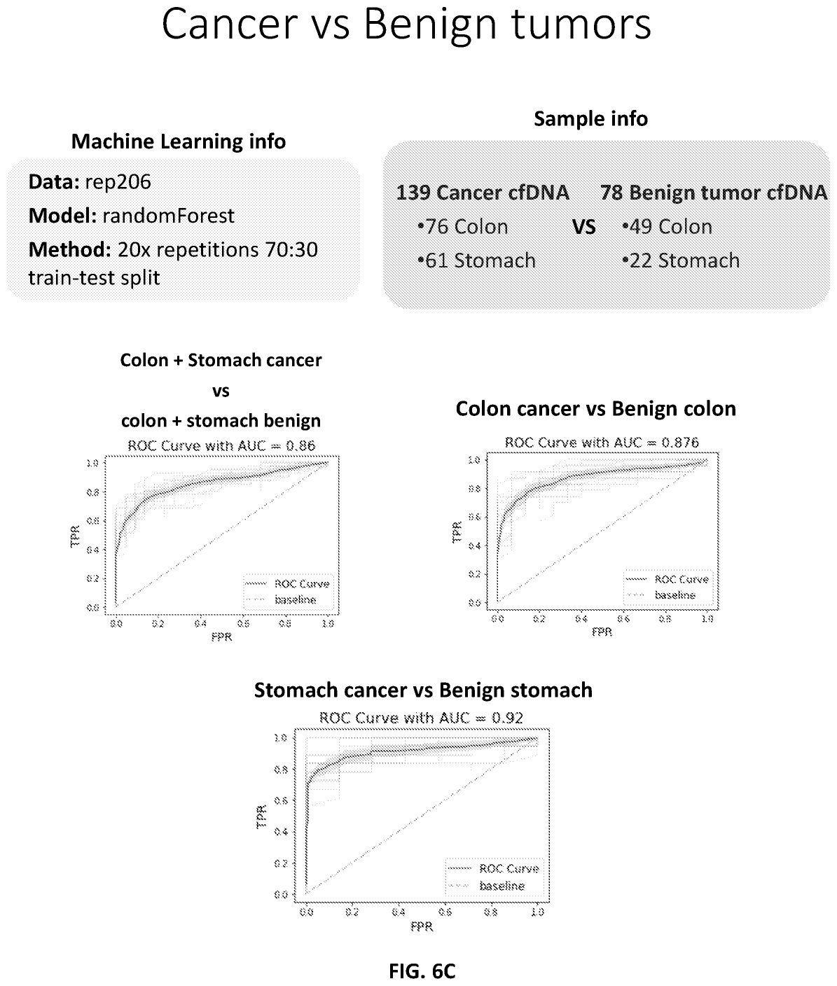

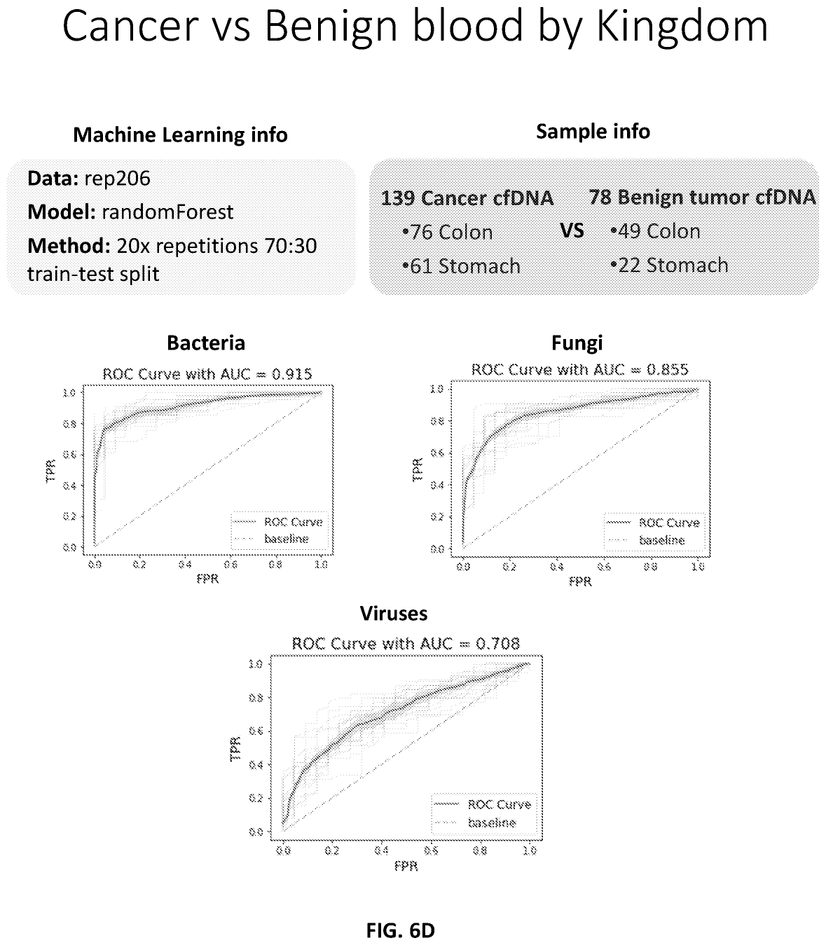

FIG. 6A shows the cell-free DNA study whence the 5-hydroxylmethylcytosine-enrichment sequencing data was drawn, and the sample types present therein. FIG. 6B shows the performance of a random forest machine learning classifier trained on 5hmC-enriched microbial nucleic acids from various cancer types and healthy individuals. ROC curves for each cancer type vs. healthy are given with the cancer type specified above each respective ROC curve. FIG. 6C shows the performance of a random forest machine learning classifier trained on 5hmC-enriched microbial nucleic acids from colon and stomach cancers as well as benign tumors from colon and stomach. FIG. 6D shows the performance of a random forest machine learning classifier trained on the same samples from FIG. 6C: in this instance, however, the microbial 5hmC feature sets were restricted to specific microbial kingdoms (i.e., bacteria, fungi, and viruses), thereby demonstrating that all three kingdoms contain 5hmC-bearing features with cancer vs, benign discriminatory power.

### Example 2: Identification of 5hmC-positive microbial genomic regions via hMeDIP-seq method

5hmC enrichment is performed using Active Motif s hMeDIP kit (#55010) as per manufacturer's protocol. Briefly. 3-5 μg of human brain DNA (Zyagen #HG0201). Pseudomonas aeruginosa strain PAO1-LAC DNA (ATCC #47085D-5), Escherichia coli strain EDL 933 DNA (ATCC #700927D-5), and Bacillus subtilis strain 168 DNA (ATCC #23857D-5) are fragmented using enzymatic digestion as per manufacturer's protocol (Roche's KAPA frag kit for enzymatic fragmentation. #07962517001). Samples are incubated for 8 minutes at 37 C and purified afterwards using AMPure XP beads (Beckman Coulter #A63881). Fragmented DNA are quantified using Qubit Ix dsDNA HS Assay Kit (ThermoFisher #Q33231), and fragmentation profile are visualized using TapeStation genomic (Agilent #5067-5365) and D1000 (Agilent #5067-5582) tapes. 100 ng of fragmented human brain gDNA and 500 ng of DNA from Pseudomonas aeruginosa, Escherichia coli, and Bacillus subtilis are incubated with 4 μg of either rabbit anti-5hmC antibody or control IgG while rotating overnight at 4 C. 10% of material (10) ng and 50 ng respectively) is reserved as input and stored at −80 C until downstream purification and analysis. 25 μL of Pierce protein A/G plus agarose beads (ThermoFisher #20423) are added to capture protein-antibody complexes by rotating samples for 2 h at room temperature, followed by washes as indicated in the manufacturer's protocol. The captured antibody-protein complexes are eluted off the beads using SDS-mediated elution. Equal volume of elution buffer is added to inputs as well. Eluted immunoprecipitated (IP) material and their respective inputs are purified using Qiagen MinElute columns. They are then subjected to qPCR-based QC analysis to assess IP efficiency, followed subsequent library preparation.

2S™ Plus DNA Library Kit (IDT #10009878) and 2S™ MID Adapter Set A+B (IDT #10009902) are used to prepare libraries as per manufacturer's protocol. Briefly, 9 and 14 PCR cycles were used to amplify inputs and IPs, respectively. Final libraries are eluted in 25 μL volume. Final libraries are quantified using KAPA library quantification kit (Roche #07960140001) and Qubit 1× dsDNA HS Assay Kit and visualized using TapeStation D1000 tape. They are sequenced paired end (150x150 8x0) on NextSeq 2000 using P3 chemistry (Illumina #20040561). Genome-wide 5hmC enrichments are computationally identified via the MeDIPS package (Lienhard, M., Grimm, C., Morkel, M., Herwig, R., & Chavez, L. (2014). MEDIPS: genome-wide differential coverage analysis of sequencing data derived from DNA enrichment experiments. Bioinformatics (Oxford. England), 30 (2), 284-286. https://doi.org/10.1093/bioinformatics/btt650) where statistically significant increases in sequencing reads at genomic loci of interest over the read number found in the non-immunoprecipitated input control are calculated and tabulated.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “epigenetic feature” is used to describe heritable and reversible chemical modifications to nucleic acids installed or removed by a cell's biochemical machinery (enzymes) as opposed to nucleic acid modifications introduced by chemical or environmental agents. This also applies to chemical modifications to viral nucleic acids produced through viral recruitment of host cell enzymatic machinery and/or through viral enzymes during the infection process.

The terms “metaepigenetic” and “metaepigenomic” are used to describe analyses that combine epigenetic data, such as nucleic acid sequencing data, derived from the analysis of nucleic acids from more than one kingdom of life. In these instances, the sequencing data is derived from nucleic acid enrichments that employed one or more epigenetic features to concentrate nucleic acids bearing the targeted epigenetic feature.

The term “epigenetic writer” is used to describe enzymes that perform the necessary biochemical reaction(s) to install a specific nucleotide modification. For example, mammalian DNA methyltransferases are ‘epigenetic writers’ that install methyl groups on select cytosine nucleotides within the genome.

The term ‘epigenetic reader’ is used to describe proteins capable of recognizing the epigenetic marks and promoting/orchestrating cellular or transcriptional events that are dependent upon recognition of the epigenetic mark in question.

The term ‘epigenetic eraser’ is used to describe enzymes that perform the necessary biochemical reaction(s) to remove a specific nucleotide modification.

The term “taxonomic abundance” is used to describe the number of sequencing reads that can be assigned to identified microbial taxa in each sample.

The term “inter-kingdom” is used to describe analyses that combine biological or molecular data or features from two or more taxonomic kingdoms of life (here, mammalian, bacterial, archaeal, fungal, and viral).

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

What is claimed:

1. A method of determining a disease of a subject, comprising:
(a) providing a biological sample of a subject;
(b) enriching one or more nucleic acid molecules of the biological sample by affinity targeting of an epigenetic feature common to the one or more nucleic acid molecules;
(c) sequencing the enriched one or more nucleic acid molecules to generate one or more nucleic acid molecule sequencing reads; and
(d) determining the disease of the subject as an output of a predictive model when the predictive model is provided the enriched one or more nucleic acid molecules as an input.

2. The method of claim 1, wherein the one or more nucleic acid molecules comprise one or more mammalian nucleic acid molecules, one or more non-mammalian nucleic acid molecules, or a combination thereof.

3. The method of claim 1, wherein the disease comprises cancer or a non-cancerous disease.

4. The method of claim 3, wherein the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

5. The method of claim 3, wherein the non-cancerous disease comprises lupus erythematosus, type 2 diabetes, chronic obstructive pulmonary disease (COPD), sarcoidosis, or any combination thereof non-cancer diseases.

6. The method of claim 1, further comprising filtering the one or more nucleic acid molecules sequencing reads to identify one or more non-mammalian sequencing reads and one or more mammalian sequencing reads.

7. The method of claim 1, wherein the epigenetic feature comprises a nucleic acid epigenetic feature.

8. The method of claim 1, wherein the epigenetic feature comprises a mammalian nucleic acid epigenetic feature or a non-mammalian nucleic acid epigenetic feature.

9. The method of claim 8, wherein the non-mammalian nucleic acid epigenetic feature comprises phosphorothioate-linked nucleotides.

10. The method of claim 1, wherein the biological sample comprises a tissue, liquid biopsy sample or a combination thereof.

11. The method of claim 1, wherein the subject is human or a non-human mammal.

12. The method of claim 2, wherein the one or more mammalian nucleic acid molecules, comprise DNA, RNA, cell-free DNA, cell-free RNA, exosomal DNA, exosomal RNA, or any combination thereof.

13. The method of claim 2, wherein the one or more non-mammalian nucleic acid molecules, comprise DNA, RNA, cell-free DNA, cell-free RNA, exosomal DNA, exosomal RNA, or any combination thereof.

14. The method of claim 7, wherein the affinity targeting of the nucleic acid epigenetic feature comprises concentrating the nucleic acid epigenetic feature.

15. The method of claim 7, wherein the nucleic acid epigenetic feature comprises methylated CpG dinucleotides pairs, unmethylated CpG dinucleotide pairs, or a combination thereof.

16. The method of claim 7, wherein the nucleic acid epigenetic feature comprises nucleobases 5-methylcytosine, 5-hydroxymethylcytosine, N4-acetylcytosine, N6-methyladenine, or any combination thereof.

17. The method of claim 1, wherein the affinity targeting utilizes specific affinity reagents to bind to the epigenetic feature.

18. The method of claim 17, wherein the specific affinity reagents comprise streptavidin, NeutrAvidin, polyclonal, monoclonal, recombinant antibodies, aptamers, recombinant epigenetic proteins, or any combination thereof.

19. The method of claim 18, wherein the recombinant epigenetic proteins comprise epigenetic readers, writers, erasers, or any combination thereof.

20. The method of claim 19, wherein the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom.

21. The method of claim 19, wherein the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom.

22. The method of claim 19, wherein the epigenetic readers comprise the microbial proteins Dam, CcrM, ModA13, SpnD39III, Dcm, JHP1050, M2.Hpy.AII, or the recombinant methyl-binding domains derived therefrom.

23. The method of claim 19, wherein the epigenetic writers and erasers are catalytically inactive.

24. The method of claim 19, wherein the epigenetic readers, writers, and erasers comprise an epitope tag.

25. The method of claim 24, wherein the epitope tag comprises a N- or C-terminal 6x-histidine tag, green fluorescent protein (MA), myc, hemagglutinin (HA), Fc fusion, molecular recognition motif, or any combination thereof.

26. The method of claim 25, wherein the molecular recognition motif comprises a birA or sortase motif.

27. The method of claim 2, further comprising concentrating the one or more mammalian and non-mammalian nucleic acid molecules by a solid support, wherein the solid support comprises immobilized complementary antibodies to the epitope tag.

28. The method of claim 17, wherein the specific affinity reagents comprise a region to recognize and bind to the epigenetic feature.

29. The method of claim 1, wherein the affinity targeting comprises incubating the biological sample with a solid support comprising a plurality of immobilized affinity agents.

30. The method of claim 29, wherein the plurality of immobilized affinity agents comprises a region that will bind to the epigenetic feature.

31. The method of claim 29, wherein the solid support comprises a magnetic bead, an agarose bead, non-magnetic latex, functionalized Sepharose, pH-sensitive polymers, or any combination thereof.

32. The method of claim 6, wherein filtering comprises filtering the one or more mammalian sequencing reads and the one or more non-mammalian sequencing reads against a genome database.

33. The method of claim 32, wherein the genome database is a human genome database.

34. The method of claim 3, wherein the predictive model is trained with one or more mammalian, one or more non-mammalian, or a combination thereof features determined from one or more subjects' one or more nucleic acid molecules of a biological sample and a corresponding disease of the one or more subjects.

35. The method of claim 34, wherein the one or more mammalian feature comprises mammalian genomic coordinates or annotated genomic loci and a number of sequencing reads associated therewith.

36. The method of claim 34, wherein the one or more mammalian feature comprises mammalian functional gene and biochemical pathway abundances.

37. The method of claim 34, wherein the one or more non-mammalian feature comprises microbial taxonomic assignments and a number of sequencing reads associated therewith.

38. The method of claim 34, wherein the one or more non-mammalian features comprises microbial functional gene and biochemical pathway abundances.

39. The method of claim 10, wherein the liquid biopsy sample comprises plasma, serum, whole blood, urine, cerebral spinal fluid, saliva, sweat, tears, exhaled breath condensate, or any combination thereof.

40. The method of claim 1, wherein the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when one or more nucleic acid molecules of the biological sample are not enriched.

41. The method of claim 1, wherein the predictive model comprises an area under the curve of at least about 0.70, at least about 0.80, at least about 0.85, at least about 0.90, or at least about 0.95 when determining the disease of the subject.

42. The method of claim 34, wherein an output of the trained predictive model comprises an analysis of a combination of the one or more mammalian features and the one or more non-mammalian features abundance.

43. The method of claim 34, wherein an input of the trained predictive model comprises epigenomic abundance information from one or more of the following kingdoms of life: mammalian, bacterial, archaeal, fungal, and/or viral.

44. The method of claim 34, wherein the predictive model is further trained with a tissue-specific location of the disease.

45. The method of claim 34, wherein the predictive model is further trained with the cancer's type, subtype, stage, prognosis, or any combination thereof.

46. The method of claim 3, wherein the predictive model outputs the cancer's type, subtype, stage, prognosis, or any combination thereof when provided the subject's nucleic acid sequencing reads of the biological sample.

47. The method of claim 3, wherein the predictive model outputs the subject's cancer therapy response.

48. The method of claim 34, wherein the trained predictive model outputs a therapy for the subject that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the subject.

49. The method of claim 34, wherein the trained predictive model outputs a longitudinal model of the subject's cancer in response to a therapy, an adjustment to a therapy to treat the subject's cancer, or a combination thereof.

50. The method of claim 34, wherein the predictive model removes contaminate non-mammalian features while selectively retaining other non-contaminate non-mammalian features.

51. The method of claim 1, wherein the enriching reduces a total of the one or more nucleic acid molecules by at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99%.

52. A method of training a predictive model, comprising:
(a) providing a biological sample of one or more subjects with a disease;
(b) enriching the biological sample of the one or more subjects by affinity targeting of an epigenetic feature common to one or more nucleic acid molecules of the biological sample;
(c) sequencing the enriched one or more nucleic acid molecules to generate one or more nucleic acid molecule sequencing reads; and
(d) training the predictive model with one or more features of the one or more nucleic acid molecule sequencing reads and the disease of the one or more subjects.

53. The method of claim 52, wherein the epigenetic feature comprises a mammalian epigenetic feature or a non-mammalian epigenetic feature.

54. The method of claim 52, wherein the one or more features comprise one or more disease features.

55. The method of claim 52, wherein the trained predictive model determines a disease of another one or more subjects that differ from the one or more subjects when the trained predictive model is provided the another one or more subjects' nucleic acid sequencing reads of a biological sample.

56. The method of claim 52, wherein the one or more nucleic acid molecules comprise one or more mammalian nucleic acid molecules, one or more non-mammalian nucleic acid molecules, or a combination thereof.

57. The method of claim 56, further comprising filtering the one or more nucleic acid sequencing reads to identify the one or more non-mammalian sequencing reads, the one or more mammalian sequencing reads, or a combination thereof.

58. The method of claim 52, wherein the epigenetic feature comprises a nucleic acid epigenetic feature.

59. The method of claim 52, wherein the biological sample comprises a tissue, liquid biopsy sample or a combination thereof.

60. The method of claim 52, wherein the one or more subjects are human or a non-human mammal.

61. The method of claim 56, wherein the one or more mammalian nucleic acid molecules comprise DNA, RNA, cell-free DNA, cell-free RNA, exosomal DNA, exosomal RNA, or any combination thereof.

62. The method of claim 56, wherein the one or more non-mammalian nucleic acid molecules comprise DNA, RNA, cell-free DNA, cell-free RNA, exosomal DNA, exosomal RNA, or any combination thereof.

63. The method of claim 58, wherein the affinity targeting of the nucleic acid epigenetic feature comprises concentrating the nucleic acid epigenetic feature.

64. The method of claim 58, wherein the nucleic acid epigenetic feature comprises methylated CpG dinucleotides pairs, unmethylated CpG dinucleotide pairs, or a combination thereof.

65. The method of claim 58, wherein the nucleic acid epigenetic feature comprises nucleobases 5-methylcytosine, 5-hydroxymethylcytosine, N4-acetylcytosine, N6-methyladenine, or any combination thereof.

66. The method of claim 52, wherein the affinity targeting utilizes specific affinity reagents to bind to the epigenetic feature.

67. The method of claim 66, wherein the specific affinity reagents comprise streptavidin, NeutrAvidin, polyclonal, monoclonal, recombinant antibodies, aptamers, recombinant epigenetic proteins, or any combination thereof.

68. The method of claim 67, wherein the recombinant epigenetic proteins comprise epigenetic readers, writers, erasers, or any combination thereof.

69. The method of claim 68, wherein the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom.

70. The method of claim 68, wherein the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom.

71. The method of claim 68, wherein the epigenetic writers and erasers are catalytically inactive.

72. The method of claim 68, wherein the epigenetic readers, writers, and erasers comprise an epitope tag.

73. The method of claim 72, wherein the epitope tag comprises a N- or C-terminal 6x-histidine tag, green fluorescent protein (MA), myc, hemagglutinin (HA), Fc fusion, molecular recognition motif, or any combination thereof.

74. The method of claim 73, wherein the molecular recognition motif comprises a birA or sortase motif.

75. The method of claim 56, further comprising concentrating the one or more mammalian and the one or more non-mammalian nucleic acid molecules by a solid support, wherein the solid support comprises immobilized complementary antibodies to the epitope tag.

76. The method of claim 66, wherein the specific affinity reagents comprise a region to recognize and bind to the epigenetic feature.

77. The method of claim 52, wherein the affinity targeting comprises incubating the biological sample with a solid support comprising a plurality of immobilized affinity agents.

78. The method of claim 77, wherein the plurality of immobilized affinity agents comprises a region that will bind to the epigenetic feature.

79. The method of claim 77, wherein the solid support comprises a magnetic bead, an agarose bead, non-magnetic latex, functionalized Sepharose, pH-sensitive polymers, or any combination thereof.

80. The method of claim 57, wherein filtering comprises filtering the one or more mammalian and non-mammalian sequencing reads against a genome database.

81. The method of claim 80, wherein the genome database is a human genome database.

82. The method of claim 52, wherein the one or more features comprise one or more mammalian features, one or more non-mammalian features, or a combination thereof features.

83. The method of claim 82, wherein the one or more mammalian features comprise mammalian genomic coordinates or annotated genomic loci and a number of sequencing reads associated therewith.

84. The method of claim 82, wherein the one or more mammalian features comprise mammalian functional gene and biochemical pathway abundances.

85. The method of claim 82, wherein the one or more non-mammalian features comprise microbial taxonomic assignments and a number of sequencing reads associated therewith.

86. The method of claim 82, wherein the one or more non-mammalian features comprises microbial functional gene and biochemical pathway abundances.

87. The method of claim 59, wherein the liquid biopsy sample comprises plasma, serum, whole blood, urine, cerebral spinal fluid, saliva, sweat, tears, exhaled breath condensate, or any combination thereof.

88. The method of claim 55, wherein the disease comprises cancer or non-cancerous disease.

89. The method of claim 55, wherein the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when one or more nucleic acid molecules of the biological sample are not enriched.

90. The method of claim 52, wherein the predictive model comprises an area under the curve of at least about 0.70, at least about 0.80, at least about 0.85, at least about 0.90, or at least about 0.95 when determining the disease of the another one or more subjects.

91. The method of claim 53, wherein the non-mammalian nucleic acid epigenetic feature comprises phosphorothioate-linked nucleotides.

92. The method of claim 68, wherein the epigenetic readers comprise the microbial proteins Dam, CcrM, ModA13, SpnD39III, Dcm, JHP1050, M2.Hpy.AII, or the recombinant methyl-binding domains derived therefrom.

93. The method of claim 82, wherein an output of the trained predictive model comprises an analysis of a combination of the one or more mammalian features and the one or more non-mammalian features abundance.

94. The method of claim 52, wherein an input of the trained predictive model comprises epigenomic abundance information from one or more of the following kingdoms of life: mammalian, bacterial, archaeal, fungal, and/or viral.

95. The method of claim 88, wherein the predictive model is further trained with a tissue-specific location of the disease.

96. The method of claim 88, wherein the predictive model is further trained with the cancer's type, subtype, stage, prognosis, or any combination thereof.

97. The method of claim 88, wherein the predictive model outputs the cancer's type, subtype, stage, prognosis, or any combination thereof when provided the another one or more subjects' nucleic acid sequencing reads of the biological sample.

98. The method of claim 55, wherein the trained predictive model outputs the another one or more subjects' cancer therapy response.

99. The method of claim 55, wherein the trained predictive model outputs a therapy for the another one or more subjects that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the another one or more subjects.

100. The method of claim 55, wherein the trained predictive model outputs a longitudinal model of the another one or more subjects' cancers in response to a therapy, an adjustment to a therapy to treat the another one or more subjects' cancer, or a combination thereof.

101. The method of claim 88, wherein the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

102. The method of claim 52, wherein the predictive model is configured to remove contaminate non-mammalian features while selectively retaining other non-contaminate non-mammalian features.

103. The method of claim 88, wherein the non-cancerous disease comprises lupus erythematosus, type 2 diabetes, chronic obstructive pulmonary disease (COPD), sarcoidosis, or any combination thereof non-cancer diseases.

104. The method of claim 52, wherein the enriching reduces a total of the one or more nucleic acid molecules by at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99%.

105. A computer system to determine a disease of a subject, comprising:
(a) one or more processors; and
(b) a non-transient computer readable storage medium including software, wherein the software comprises executable instruction that, as a result of execution, cause the one or more processors of the computer system to:
(i) receive a subject's one or more nucleic acid molecule sequencing reads of one or more nucleic acid molecules of a biological sample, wherein the one or more nucleic acid molecules are enriched by affinity targeting of an epigenetic feature common to the one or more nucleic acid molecules; and
(ii) determine a disease of the subject as an output of a predictive model when the predictive model is provided the one or more nucleic acid molecule sequencing.

106. The method of claim 105, wherein the one or more nucleic acid molecules comprise one or more mammalian nucleic acid molecules, one or more non-mammalian nucleic acid molecules, or a combination thereof.

107. The method of claim 105, wherein the disease comprises cancer or a non-cancerous disease.

108. The method of claim 107, wherein the cancer comprises acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof.

109. The method of claim 107, wherein the non-cancerous disease comprises lupus erythematosus, type 2 diabetes, chronic obstructive pulmonary disease (COPD), sarcoidosis, or any combination thereof non-cancer diseases.

110. The method of claim 105, wherein the executable instruction further comprise filter the one or more nucleic acid molecules sequencing reads to identify one or more non-mammalian sequencing reads and one or more mammalian sequencing reads.

111. The method of claim 105, wherein the epigenetic feature comprises a nucleic acid epigenetic feature.

112. The method of claim 105, wherein the epigenetic feature comprises a mammalian nucleic acid epigenetic feature or a non-mammalian nucleic acid epigenetic feature.

113. The method of claim 112, wherein the non-mammalian nucleic acid epigenetic feature comprises phosphorothioate-linked nucleotides.

114. The method of claim 105, wherein the biological sample comprises a tissue, liquid biopsy sample or a combination thereof.

115. The method of claim 105, wherein the subject is human or a non-human mammal.

116. The method of claim 106, wherein the one or more mammalian nucleic acid molecules, comprise DNA, RNA, cell-free DNA, cell-free RNA, exosomal DNA, exosomal RNA, or any combination thereof.

117. The method of claim 106, wherein the one or more non-mammalian nucleic acid molecules, comprise DNA, RNA, cell-free DNA, cell-free RNA, exosomal DNA, exosomal RNA, or any combination thereof.

118. The method of claim 111, wherein the affinity targeting of the nucleic acid epigenetic feature comprises concentrating the nucleic acid epigenetic feature.

119. The method of claim 111, wherein the nucleic acid epigenetic feature comprises methylated CpG dinucleotides pairs, unmethylated CpG dinucleotide pairs, or a combination thereof.

120. The method of claim 111, wherein the nucleic acid epigenetic feature comprises nucleobases 5-methylcytosine, 5-hydroxymethylcytosine, N4-acetylcytosine, N6-methyladenine, or any combination thereof.

121. The method of claim 105, wherein the affinity targeting utilizes specific affinity reagents to bind to the epigenetic feature.

122. The method of claim 121, wherein the specific affinity reagents comprise streptavidin, NeutrAvidin, polyclonal, monoclonal, recombinant antibodies, aptamers, recombinant epigenetic proteins, or any combination thereof.

123. The method of claim 122, wherein the recombinant epigenetic proteins comprise epigenetic readers, writers, erasers, or any combination thereof.

124. The method of claim 123, wherein the epigenetic readers comprise recombinant methyl-CpG binding proteins Mecp2, Mbd1-6, SETDB1, SETDB2, TIP5/BAZ2A, Zbtb38, Kaiso, Zbtb4, Np95, Np97 or the recombinant methyl-binding domains derived therefrom.

125. The method of claim 123, wherein the epigenetic readers comprise recombinant zinc finger CXXC domain-containing proteins KDM2A, KDM2A, KDM2B, FBXL19, CFP1, DNMT1, MLL1, MLL2, MDB1, TET1, TET3, IDAX, CXXC5, CGBP, or the recombinant CXXC domains derived therefrom.

126. The method of claim 123, wherein the epigenetic readers comprise the microbial proteins Dam, CcrM, ModA13, SpnD39III, Dem, JHP1050, M2.Hpy.AII, or the recombinant methyl-binding domains derived therefrom.

127. The method of claim 123, wherein the epigenetic writers and erasers are catalytically inactive.

128. The method of claim 123, wherein the epigenetic readers, writers, and erasers comprise an epitope tag.

129. The method of claim 128, wherein the epitope tag comprises a N- or C-terminal 6x-histidine tag, green fluorescent protein (MA), myc, hemagglutinin (HA), Fc fusion, molecular recognition motif, or any combination thereof.

130. The method of claim 129, wherein the molecular recognition motif comprises a birA or sortase motif.

131. The method of claim 106, further comprising concentrating the one or more mammalian and non-mammalian nucleic acid molecules by a solid support, wherein the solid support comprises immobilized complementary antibodies to the epitope tag.

132. The method of claim 121, wherein the specific affinity reagents comprise a region to recognize and bind to the epigenetic feature.

133. The method of claim 105, wherein the affinity targeting comprises incubating the biological sample with a solid support comprising a plurality of immobilized affinity agents.

134. The method of claim 133, wherein the plurality of immobilized affinity agents comprises a region that will bind to the epigenetic feature.

135. The method of claim 133, wherein the solid support comprises a magnetic bead, an agarose bead, non-magnetic latex, functionalized Sepharose, pH-sensitive polymers, or any combination thereof.

136. The method of claim 110, wherein filtering comprises filtering the one or more mammalian sequencing reads and the one or more non-mammalian sequencing reads against a genome database.

137. The method of claim 136, wherein the genome database is a human genome database.

138. The method of claim 107, wherein the predictive model is trained with one or more mammalian, one or more non-mammalian, or a combination thereof features determined from one or more subjects' one or more nucleic acid molecules of a biological sample and a corresponding disease of the one or more subjects.

139. The method of claim 138, wherein the one or more mammalian feature comprises mammalian genomic coordinates or annotated genomic loci and a number of sequencing reads associated therewith.

140. The method of claim 138, wherein the one or more mammalian feature comprises mammalian functional gene and biochemical pathway abundances.

141. The method of claim 138, wherein the one or more non-mammalian feature comprise microbial taxonomic assignments and a number of sequencing reads associated therewith.

142. The method of claim 138, wherein the one or more non-mammalian features comprise microbial functional gene and biochemical pathway abundances.

143. The method of claim 114, wherein the liquid biopsy sample comprises plasma, serum, whole blood, urine, cerebral spinal fluid, saliva, sweat, tears, exhaled breath condensate, or any combination thereof.

144. The method of claim 105, wherein the predictive model's accuracy of determining the disease is increased by at least about 10%, at least about 20%, at least about 30%, at least about 40%, at least about 50%, at least about 60%, at least about 70%, at least about 80%, at least about 85%, at least about 90%, or at least about 95% when the one or more nucleic acid molecules of the biological sample are enriched compared to when the one or more nucleic acid molecules of the biological sample are not enriched.

145. The method of claim 105, wherein the predictive model comprises an area under the curve of at least about 0.70, at least about 0.80, at least about 0.85, at least about 0.90, or at least about 0.95 when determining the disease of the subject.

146. The method of claim 138, wherein an output of the trained predictive model comprises an analysis of a combination of the one or more mammalian features and the one or more non-mammalian features abundance.

147. The method of claim 105, wherein an input of the trained predictive model comprises epigenomic abundance information from one or more of the following kingdoms of life: mammalian, bacterial, archaeal, fungal, and/or viral.

148. The method of claim 138, wherein the predictive model is further trained with a tissue-specific location of the disease.

149. The method of claim 138, wherein the predictive model is further trained with the cancer's type, subtype, stage, prognosis, or any combination thereof.

150. The method of claim 107, wherein the predictive model outputs the cancer's type, subtype, stage, prognosis, or any combination thereof when provided the subject's nucleic acid sequencing reads of the biological sample.

151. The method of claim 107, wherein the predictive model outputs the subject's cancer therapy response.

152. The method of claim 107, wherein the trained predictive model outputs a therapy for the subject that results in at least about 5%, at least about 10%, at least about 20%, at least about 30%, at least about 40%, at least about 50%, at least about 60%, at least about 70%, at least about 80%, at least about 90%, or at least about 95% reduction in cancerous regions of the subject.

153. The method of claim 107, wherein the trained predictive model outputs a longitudinal model of the subject's cancer in response to a therapy, an adjustment to a therapy to treat the subject's cancer, or a combination thereof.

154. The method of claim 105, wherein the predictive model removes contaminate non-mammalian features from the one or more sequencing reads while selectively retaining other non-contaminate non-mammalian features.

155. The method of claim 105, wherein the enriched nucleic acids comprise a reduction of at least about 5%, at least about 10%, at least about 15%, at least about 20%, at least about 30%, at least about 40%, at least about 50%, at least about 60%, at least about 70%, at least about 80%, at least about 90%, at least about 95%, at least about 97%, at least about 98%, or at least about 99% of the one or more nucleic acid molecules prior to enrichment.
